(19) United States
(12) Patent Application Publication
(10) Pub. No.: US 2024/0398797 A1
(43) Pub. Date: Dec. 5, 2024

(54) COMBINATION THERAPIES FOR METASTATIC CASTRATION-RESISTANT PROSTATE CANCER

(71) Applicants: Janssen Pharmaceutica NV, Beerse (BE); ESSA Pharma Inc., Vancouver (CA)

(72) Inventors: Won KIM, Raritan, NJ (US); Peter VIRSIK, Portola Valley, CA (US)

(21) Appl. No.: 18/688,073

(22) PCT Filed: Sep. 1, 2022
(86) PCT No.: PCT/EP2022/074400

§ 371 Date: Feb. 29, 2024

Related U.S. Application Data
(60) Provisional application No. 63/239,616, filed on Sep. 1, 2021.

(51) Int. Cl. A61K 31/505; A61K 31/4439; A61K 31/573; A61K 31/58

(52) U.S. Cl. CPC A61K 31/505; A61K 31/4439; A61K 31/573; A61K 31/58

(57) ABSTRACT

Provided herein are the compound of formula I in combination with (a) abiraterone acetate plus prednisone or (b) apalutamide for use in methods of treating metastatic castration-resistant prostate cancer (mCRPC) in a male human. Also provided herein are the compound of formula I in combination with (a) abiraterone acetate plus prednisone or (b) apalutamide for use in methods of achieving a composite response rate in a male human.

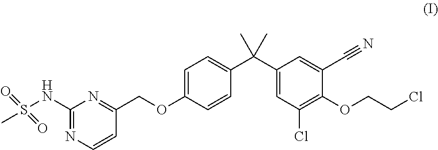 [SMILES: CC(C)(c1ccc(OCc2ccnc(NS(C)(=O)=O)n2)cc1)c1cc(Cl)c(OCCCl)c(C#N)c1]

### TECHNICAL FIELD

Disclosed herein are methods of treating metastatic castration-resistant prostate cancer (mCRPC) in a male human by administering the compound of formula I in combination with (a) abiraterone acetate and a corticosteroid or (b) apalutamide. Also disclosed herein are methods of achieving a composite response rate in a male human comprising administering the compound of formula I in combination with (a) abiraterone acetate plus a corticosteroid or (b) apalutamide.

### BACKGROUND

Prostate cancer is the most common non-cutaneous malignancy in men and the second leading cause of death in men from cancer in the western world. Prostate cancer results from the uncontrolled growth of abnormal cells in the prostate gland. Once a prostate cancer tumor develops, androgens, such as testosterone, promote prostate cancer tumor growth. Not all prostate cancer is the same. It ranges from cancer confined to the prostate gland, i.e., localized, to cancer that has spread outside of the prostate to the lymph nodes, bones, or other parts of the body, i.e., metastatic. At its early stages, localized prostate cancer is often treated with local therapy including, for example, surgical removal of the prostate gland and radiotherapy. However, when local therapy fails to cure prostate cancer, as it does in up to a third of men, the disease progresses into incurable metastatic disease (i.e., disease in which the cancer has spread from one part of the body to other parts).

Suppressing the androgen axis is the backbone of treatment of advanced prostate cancer. Androgen deprivation therapy (ADT) reduces testosterone to castrate levels (≤50 ng/mL) through medical or surgical approaches. ADT is a lifelong therapy of prostate cancer treatment for men with metastatic disease. Almost all prostate cancers initially respond to ADT, but in many, the response is short-lived with such men having prostate cancer that is castration resistant (CRPC). Various mechanisms are thought to be involved in castration resistance, including amplification, overexpression or mutation of the androgen receptor (AR), constitutive activation of AR, alternative splicing events, intra-tumoral androgen synthesis or androgen synthesis by the adrenal glands, activation of other ligands, and proliferation of prostate tumor cells independent of androgen.

Androgen signaling inhibitors (ASIs) target the androgen-signaling pathway through a variety of mechanisms. Abiraterone acetate (AA) is a prodrug of abiraterone, which is an irreversible, highly selective Cytochrome p450 (CYP) 17 inhibitor that targets 17α-hydroxylase and C17,20-lyase activities resulting in reduced intratumoral production of androgens reducing as well their synthesis in the adrenal glands and the testes. Apalutamide (APA) targets the ligand binding domain of the AR, which inhibits the androgen receptor and prohibits nuclear translocation, DNA binding and transcription without AR agonistic effects. More recently, agents that target the AR N-terminal domain (NTD), e.g., the compound of formula I, are being investigated as a different therapeutic approach to treating prostate cancer. Accordingly, a need exists for the identification and development of methods for the treatment of prostate cancer that achieve complete blockage of the androgen receptor by combining ASIs with AR NTD inhibitors.

### SUMMARY

Described herein are methods of treating metastatic castration-resistant prostate cancer (mCRPC) in a male human, said methods comprising, consisting of, or consisting essentially of, administering a therapeutically effective amount of the compound of formula I and a therapeutically effective amount of abiraterone acetate plus a therapeutically effective amount of a corticosteroid to said male human.

In some embodiments, prior to administration of the compound of formula I and abiraterone acetate plus a corticosteroid, the male human has been administered docetaxel. In certain embodiments, prior to administration of the compound of formula I and abiraterone acetate plus a corticosteroid, the male human has been diagnosed with non-metastatic castration-resistant prostate cancer, metastatic castration-resistant prostate cancer, metastatic castration-sensitive prostate cancer, or locally advanced prostate cancer.

In some embodiments, abiraterone acetate is administered orally to the male human at a dose of about 500 mg per day to about 1000 mg per day. In certain embodiments, abiraterone acetate is administered orally to the male human at a dose of about 1000 mg per day.

In some embodiments, the corticosteroid is prednisone, a prednisolone, or dexamethasone. In certain embodiments, the corticosteroid is administered to the male human at a dose of about 5 mg twice per day.

Also described herein are methods of treating mCRPC in a male human, said methods comprising, consisting of, or consisting essentially of, administering a therapeutically effective amount of the compound of formula I and a therapeutically effective amount of apalutamide to said male human. In some embodiments, apalutamide is administered orally to the male human at a dose of about 240 mg per day.

In some embodiments, the compound of formula I is administered orally to the male human at a dose of from about 600 mg per day to about 1000 mg per day. In certain embodiments, the compound of formula I is administered orally to the male human at a dose of: (a) about 600 mg per day; (b) about 800 mg per day; or (c) about 1000 mg per day.

In some embodiments, prior to administration of the compound of formula I and apalutamide, the male human has been administered docetaxel. In certain embodiments, prior to administration of the compound of formula I and apalutamide, the male human has been diagnosed with non-metastatic castration-resistant prostate cancer, metastatic castration-resistant prostate cancer, metastatic castration-sensitive prostate cancer, or locally advanced prostate cancer.

In some embodiments, administration provides a composite response rate in the male human In certain embodiments, the composite response rate comprises a PSA90, an objective response rate, or both. In certain embodiments, the composite response rate is at 12 weeks post administration.

In certain embodiments, the treatment is a therapeutically effective amount of the compound of formula I in combination with (a) is a therapeutically effective amount of abiraterone acetate plus is a therapeutically effective amount of a corticosteroid or (b) is a therapeutically effective amount of apalutamide each (a) and (b) in combination with androgen deprivation therapy. In some embodiments, the androgen deprivation therapy consists of bilateral orchiectomy or gonadotropin-releasing hormone agonists or antagonists.

In certain embodiments, administration of the compound of formula I is further in combination with androgen deprivation therapy. In some embodiments, the androgen deprivation therapy consists of bilateral orchiectomy or gonadotropin-releasing hormone agonists or antagonists.

Further described herein are methods of achieving a composite response rate in a male human with mCRPC, said methods comprising, consisting of, or consisting essentially of, administering a therapeutically effective amount of compound of formula I and a therapeutically effective amount of abiraterone acetate plus a therapeutically effective amount of a corticosteroid to said male human.

Still further described herein are methods achieving a composite response rate in a male human with mCRPC, said methods comprising, consisting of, or consisting essentially of, administering a therapeutically effective amount of compound of formula I and apalutamide to said male human. In certain embodiments, the composite response rate comprises a PSA90, an objective response rate, or both. In certain embodiments, the composite response rate is at 12 weeks post administration.

### THE DETAILED DESCRIPTION OF ILLUSTRATIVE EMBODIMENTS

It is to be appreciated that certain features of the invention which are, for clarity, described herein in the context of separate embodiments may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, although an embodiment may be described as part of a series of steps or part of a more general structure, each said step may also be considered an independent embodiment in itself, combinable with others.

The transitional terms “comprising,” “consisting essentially of,” and “consisting” are intended to connote their generally in accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of excludes any element, step, or ingredient not specified in the claim; and (iii) “consisting essentially of limits the scope of a claim to the specified materials or steps “and those that do not materially affect the basic and novel characteristic(s)” of the claimed invention. More specifically, the basic and novel characteristics relates to the ability of the method to provide at least one of the benefits described herein, including but not limited to the ability to improve the survivability of the male human population relative to the survivability of the comparative male human population described elsewhere herein.

Embodiments described in terms of the phrase “comprising” (or its equivalents), also provide, as embodiments, those which are independently described in terms of “consisting of and “consisting essentially of.”

When a value is expressed as an approximation by use of the descriptor “about,” it will be understood that the particular value forms another embodiment. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about.” In other cases, the gradations used in a series of values may be used to determine the intended range available to the term “about” for each value. Where present, all ranges are inclusive and combinable. That is, references to values stated in ranges include every value within that range.

If not otherwise specified, the term “about” signifies a variance of ±10% of the associated value, but additional embodiments include those where the variance may be ±5%, ±15%, ±20%, ±25%, or ±50%.

It is to be appreciated that certain features of the invention which are, for clarity, described herein in the context of separate embodiments, may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, while an embodiment may be described as part of a series of steps or part of a more general structure, each said step may also be considered an independent embodiment in itself, combinable with others.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

The present invention may be understood more readily by reference to the following description taken in connection with the accompanying Drawing and Examples, all of which form a part of this disclosure. It is to be understood that this invention is not limited to the specific products, methods, conditions or parameters described or shown herein, and that the terminology used herein is for the purpose of describing particular embodiments by way of example only and is not intended to be limiting of any claimed invention. Similarly, unless specifically otherwise stated, any description as to a possible mechanism or mode of action or reason for improvement is meant to be illustrative only, and the invention herein is not to be constrained by the correctness or incorrectness of any such suggested mechanism or mode of action or reason for improvement. Throughout this text, it is recognized that the descriptions refer to various compounds, compositions and methods of using said compounds and compositions. That is, where the disclosure describes or claims a feature or embodiment associated with a composition or a method of using a composition, it is appreciated that such a description or claim is intended to extend these features or embodiment to embodiments in each of these contexts (i.e., compositions and methods of using).

The androgen receptor (AR) is a member of the steroid and nuclear receptor superfamily. Among this large family of proteins, only five vertebrate steroid receptors are known and include the androgen receptor, estrogen receptor, progesterone receptor, glucocorticoid receptor, and mineralocorticoid receptor. AR is a soluble protein that functions as an intracellular transcriptional factor. AR function is regulated by the binding of androgens, which initiates sequential conformational changes of the receptor that affect receptor-protein interactions and receptor-DNA interactions.

AR is mainly expressed in androgen target tissues, such as the prostate, skeletal muscle, liver, and central nervous system (CNS), with the highest expression level observed in the prostate, adrenal gland, and epididymis. AR can be activated by the binding of endogenous androgens, including testosterone and 5-dihydrotestosterone (5a-DHT).

The androgen receptor (AR), located on Xql 1-12, is a 110 kD nuclear receptor that, upon activation by androgens, mediates transcription of target genes that modulate growth and differentiation of prostate epithelial cells. Similar to the other steroid receptors, unbound AR is mainly located in the cytoplasm and associated with a complex of heat shock proteins (HSPs) through interactions with the ligand-binding domain. Upon agonist binding, AR goes through a series of conformational changes: the heat shock proteins dissociate from AR, and the transformed AR undergoes dimerization, phosphorylation, and translocation to the nucleus, which is mediated by the nuclear localization signal. Recruitment of other transcription co-regulators (including co-activators and co-repressors) and transcriptional machinery further ensures the transactivation of AR-regulated gene expression. All of these processes are initiated by the ligand-induced conformational changes in the ligand-binding domain.

AR signaling is crucial for the development and maintenance of male reproductive organs including the prostate gland, as genetic males harboring loss of function AR mutations and mice engineered with AR defects do not develop prostates or prostate cancer. This dependence of prostate cells on AR signaling continues even upon neoplastic transformation. Androgen depletion (such as using GnRH agonists) continues to be the mainstay of prostate cancer treatment. However, androgen depletion is usually effective for a limited duration and prostate cancer evolves to regain the ability to grow despite low levels of circulating androgens.

Castration resistant prostate cancer (CRPC) is a lethal phenotype and almost all of patients will die from prostate cancer. Interestingly, while a small minority of CRPC does bypass the requirement for AR signaling, the vast majority of CRPC, though frequently termed “androgen independent prostate cancer” or “hormone refractory prostate cancer,” retains its lineage dependence on AR signaling.

Prostate cancer is the second most common cause of cancer death in men in the US, and approximately one in every six American men will be diagnosed with the disease during his lifetime. Treatment aimed at eradicating the tumor is unsuccessful in 30% of men, who develop recurrent disease that is usually manifest first as a rise in plasma prostate-specific antigen (PSA) followed by spread to distant sites. Given that prostate cancer cells depend on androgen receptor (AR) for their proliferation and survival, these men are treated with agents that block production of testosterone (e.g., GnRH agonists), alone or in combination with anti-androgens (e.g., bicalutamide), which antagonize the effect of any residual testosterone on AR. The approach is effective as evidenced by a drop in PSA and regression of visible tumor (if present) in some patients; however, this is followed by regrowth as a castration resistant prostate cancer (CRPC) to which most patients eventually succumb. Recent studies on the molecular basis of CRPC have demonstrated that CRPC continues to depend on AR signaling and that a key mechanism of acquired resistance is an elevated level of AR protein. AR targeting agents with activity in castration sensitive and castration resistant prostate cancer have great promise in treating this lethal disease.

The course of prostate cancer from diagnosis to death is best categorized as a series of clinical states based on the extent of disease, hormonal status, and absence or presence of detectable metastases: localized disease, rising levels of prostate-specific antigen (PSA) after radiation therapy or surgery with no detectable metastases, and clinical metastases in the non-castrate or castrate state. Although surgery, radiation, or a combination of both can be curative for patients with localized disease, a significant proportion of these patients have recurrent disease as evidenced by a rising level of PSA, which can lead to the development of metastases, especially in the high-risk group—a transition to the lethal phenotype of the disease.

Androgen depletion is the standard treatment with a generally predictable outcome: decline in PSA, a period of stability in which the tumor does not proliferate, followed by rising PSA and regrowth as castration-resistant disease. Molecular profiling studies of castration-resistance prostate cancers commonly show increased androgen receptor (AR) expression, which can occur through AR gene amplification or other mechanisms.

Anti-androgens are useful for the treatment of prostate cancer during its early stages. However, prostate cancer often advances to a castration resistant state in which the disease progresses in the presence of continued androgen ablation or anti-androgen therapy. Instances of antiandrogen withdrawal syndrome have also been reported after prolonged treatment with anti-androgens. Antiandrogen withdrawal syndrome is commonly observed clinically and is defined in terms of the tumor regression or symptomatic relief observed upon cessation of antiandrogen therapy. AR mutations that result in receptor promiscuity and the ability of these anti-androgens to exhibit agonist activity might at least partially account for this phenomenon. For example, hydroxyflutamide and bicalutamide act as AR agonists in T877A and W741L/W741C AR mutants, respectively.

In the setting of prostate cancer cells that were rendered castration resistant via overexpression of AR, it has been demonstrated that certain anti-androgen compounds, such as bicalutamide, have a mixed antagonist/agonist profile. This agonist activity helps to explain a clinical observation, called the anti-androgen withdrawal syndrome, whereby about 30% of men who progress on AR antagonists experience a decrease in serum PSA when therapy is discontinued.

### Prostate Cancer Stages

In the early stages of prostate cancer, the cancer is localized to the prostate. In these early stages, treatment typically involves either surgical removal of the prostate or radiation therapy to the prostate or observation only with no active intervention therapy in some patients. In the early stages where the prostate cancer is localized and requires intervention, surgery or radiation therapy are curative by eradicating the cancerous cells. About 30% of the time these procedures fail, and the prostate cancer continues to progress, as typically evidenced by a rising PSA level. Men whose prostate cancer has progressed following these early treatment strategies are said to have advanced or recurrent prostate cancer.

Because prostate cancer cells depend on the androgen receptor (AR) for their proliferation and survival, men with advanced prostate cancer are treated with agents that block the production of testosterone (e.g., GnRH agonists), alone or in combination with anti-androgens (e.g., bicalutamide), which antagonize the effect of any residual testosterone on AR. These treatments reduce serum testosterone to castrate levels, which generally slows disease progression for a period of time. The approach is effective as evidenced by a drop in PSA and the regression of visible tumors in some patients. Eventually, however, this is followed by regrowth referred to as castration-resistant prostate cancer (CRPC), to which most patients eventually succumb. Castration-resistant prostate cancer (CRPC) is categorized as non-metastatic or metastatic, depending on whether or not the prostate cancer has metastasized to other parts of the body.

In some embodiments, men with non-metastatic CRPC are characterized as having the following:
1. Histologically or cytologically confirmed adenocarcinoma of the prostate without neuroendocrine differentiation or small cell features, with high risk for development of metastases.
2 Castration-resistant prostate cancer demonstrated during continuous androgen deprivation therapy (ADT)/post orchiectomy. For example, defined as 3 consecutive rises of PSA, 1 week apart, resulting in two 50% increases over the nadir, with the last PSA>2 ng/mL.
3. Maintain castrate levels of testosterone (<50 ng/dL [1.72 nmol/L]) within 4 weeks of randomization and throughout the study.
4. Absence of distant metastasis by bone scan, CT or MRI scans.

### Anti-Androgens

As used herein, the term “anti-androgen” carries its generally accepted meaning and may refer to a group of hormone receptor antagonist compounds that are capable of preventing or inhibiting the biologic effects of androgens on normally responsive tissues in the body. In some embodiments, an anti-androgen is a small molecule. In some embodiments, an anti-androgen is an AR antagonist. In some embodiments, an anti-androgen is an AR full antagonist. In some embodiments, an anti-androgen is a first-generation anti-androgen. In some embodiments, an anti-androgen is a second-generation anti-androgen.

As used herein, the term “AR antagonist”, “AR inhibitor”, and “AR signaling inhibitor” are used interchangeably herein and refer to an agent that inhibits or reduces at least one activity of an AR polypeptide. Exemplary AR activities include, but are not limited to, co-activator binding, DNA binding, ligand binding, or nuclear translocation.

An exemplary androgen receptor inhibitor is 4-[7-(6-cyano-5-trifluoromethylpyridin-3-yl)-8-oxo-6-thioxo-5,7-diazaspiro [3.4] oct-5-yl]-2-fluoro-N-methylbenzamide (also known as apalutamide, ARN-509, or JNJ-56021927; CAS No. 956104-40-8).

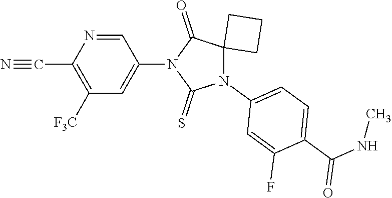 [SMILES: CNC(=O)c1ccc(N2C(=S)N(c3cnc(C#N)c(C(F)(F)F)c3)C(=O)C23CCC3)cc1F]

4-[7-(6-cyano-5-trifluoromethylpyridin-3-yl)-8-oxo-6-thioxo-5,7-diazaspiro [3.4] oct-5-yl]-2-fluoro-N-methylbenzamide (apalutamide)

Apalutamide is an androgen receptor inhibitor that binds directly to the ligand-binding domain of AR, impairing nuclear translocation, AR binding to DNA and AR target gene modulation, thereby inhibiting tumor growth and promoting apoptosis. Apalutamide binds AR with greater affinity than bicalutamide and induces partial or complete tumor regression in non-castrate hormone-sensitive and bicalutamide-resistant human prostate cancer xenograft models. Apalutamide lacks the partial agonist activity seen with bicalutamide in the context of AR overexpression. “Safety and Antitumor Activity of Apalutamide (ARN-509) in Metastatic Castration-Resistant Prostate Cancer with and without Prior Abiraterone Acetate and Prednisone” (Rathkopf, D. et al. Clin Cancer Res. 2017; 23 (14): 3544-3551) relates to the use of apalutamide as a treatment for metastatic castration-sensitive prostate cancer.

The compound of formula I (N-(4-((4-(2-(3-chloro-4-(2-chloroethoxy)-5-cyanophenyl) propan-2-yl) phenoxy) methyl) pyrimidin-2-yl) methanesulfonamideN-(4-((4-(2-(3-chloro-4-(2-chloroethoxy)-5-cyanophenyl) propan-2-yl) phenoxy) methyl) pyrimidin-2-yl) methanesulfonamide) is an orally available and selective inhibitor of N-terminal domain of the AR and has the structure as follows:

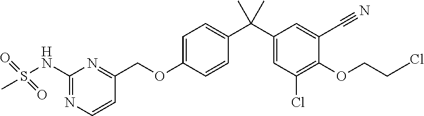 [SMILES: CCCOc1c(Cl)cc(C(C)(C)c2ccc(OCc3ccnc(NS(C)(=O)=O)n3)cc2)cc1C#N ; Cl]

(the compound of formula I)
The compound of formula I is disclosed in U.S. Pat. No. 20,210,332016A1 and WO2020/081999. In WO2020/081999 the compound of formula I is example A109.

### Certain Terminology

The terms used herein carry their normally accepted meaning, but for avoidance of doubt, some of the definitions are provided herein.

The term “cancer” as used herein refers to an abnormal growth of cells which tend to proliferate in an uncontrolled way and, in some cases, to metastasize (spread).

The term “prostate cancer” as used herein refers to histologically or cytologically confirmed adenocarcinoma of the prostate.

The term “androgen-deprivation therapy (ADT)” refers to the reduction of androgen levels in a prostate cancer patient to castrated levels of testosterone (<50 ng/dL). Such treatments can include orchiectomy or the use of gonadotropin-releasing hormone agonists or antagonists. ADT includes surgical castration (orchiectomy) and/or the administration of luteinizing hormone-releasing hormone (“LHRH”)/gonadotropin-releasing hormone (GnRH) agonists or antagonists to a human. Examples of GnRH agonist or antagonist is or comprises leuprolide, buserelin, naferelin, histrelin, goserelin, deslorelin, degarelix, ozarelix, ABT-620 (elagolix), TAK-385 (relugolix), EP-100, KLH-2109 or triptorelin. In certain embodiments, examples of GnRH agonists include goserelin acetate, histrelin acetate, leuprolide acetate, and triptorelin pamoate.

The term “locally advanced prostate cancer” refers to prostate cancer where all actively cancerous cells appear to be confined to the prostate and the associated organs or neighbor organs (e.g., seminal vesicle, bladder neck, and rectal wall).

The term “high-risk localized prostate cancer” refers to locally advanced prostate cancer that has a probability of developing metastases or recurrent disease after primary therapy with curative intent. In some embodiments, high risk for development of metastases is defined as prostate specific antigen doubling time (PSADT)<20 months, <19 months, <18 months, <17 months, <16 months, <15 months, <14 months, <13 months, <12 months, or <11 months, <10 months, <9 months, <8 months, <7 months, <6 months, <5 months, <4 months, <3 months, <2 months, or <1 month. In some embodiments, high risk for development of metastases is defined as prostate specific antigen doubling time (PSADT)<10 months. In some embodiments, high risk for development of metastases is defined as having a high Gleason score or bulky tumor.

For the avoidance of doubt, the terms “castration-sensitive prostate cancer” and “hormone-sensitive prostate cancer” are equivalent and are used interchangeably.

The terms “castration-sensitive prostate cancer” and “hormone-sensitive prostate cancer” refer to cancer that is responsive to ADT either as localized disease, biochemical relapse or in the metastatic setting.

The terms “metastatic castration-sensitive prostate cancer” and “metastatic hormone-sensitive prostate cancer” refers to cancer that has spread (metastasized) to other areas of the body, e.g., the bone, lymph nodes or other parts of the body in a male, and that is responsive to ADT.

The terms “non-metastatic castration-sensitive prostate cancer” refers to cancer that has not spread (metastasized) from the prostate in a male, and that is responsive to ADT. In some embodiments, non-metastatic castration-sensitive prostate cancer is assessed with bone scan and computed tomography (CT) or magnetic resonance imaging (MRI) scans. The term “CRPC” as used herein refers to castration-resistant prostate cancer. CRPC is prostate cancer that continues to grow despite the suppression of male hormones that fuel the growth of prostate cancer cells.

The term “metastatic castration-resistant prostate cancer” refers to castration-resistant prostate cancer that has metastasized to other parts of the human body.

Metastatic castration-sensitive prostate cancer (CSPC) refers to prostate cancer that still responds to testosterone suppression therapy.

The term “NM-CRPC” as used herein refers to non-metastatic castration-resistant prostate cancer. In some embodiments, NM-CRPC is assessed with bone scan and computed tomography (CT) or magnetic resonance imaging (MRI) scans.

The term “high risk NM-CRPC” refers to probability of a man with NM-CRPC developing metastases. In some embodiments, high risk for development of metastases is defined as prostate specific antigen doubling time (PSADT)<20 months, <19 months, <18 months, <17 months, <16 months, <15 months, <14 months, <13 months, <12 months, or <11 months, <10 months, <9 months, <8 months, <7 months, <6 months, <5 months, <4 months, <3 months, <2 months, or <1 month. In some embodiments, high risk for development of metastases is defined as prostate specific antigen doubling time (PSADT)<10 months. In some embodiments, high risk for development of metastases is defined as having local-regional recurrence (e.g. primary tumor bed, bladder neck, anastomotic area, pelvic lymph nodes).

The terms “co-administration”, “in combination”, or the like, as used herein, encompass administration of the selected therapeutic agents to a single patient, and are intended to include treatment regimens in which the agents are administered by the same or different route of administration or at the same or different time.

The term “pharmaceutical combination” as used herein, means a product that results from the mixing or combining of more than one active ingredient and includes both fixed and non-fixed combinations of the active ingredients. The term “fixed combination” means that the active ingredients, e.g., apalutamide and a co-agent, are both administered to a patient simultaneously in the form of a single unit or single dosage form. The term “non-fixed combination” means that the active ingredients, e.g., apalutamide and a co-agent, are administered to a patient as separate units or separate dosage forms, either simultaneously, concurrently or sequentially with no specific intervening time limits, wherein such administration provides safe and effective levels of the two active ingredients in the body of the human male. The latter also applies to cocktail therapy, e.g., the administration of three or more active ingredients.

The term “continuous daily dosing schedule” refers to the administration of a particular therapeutic agent without any drug holidays from the particular therapeutic agent. In some embodiments, a continuous daily dosing schedule of a particular therapeutic agent comprises administration of a particular therapeutic agent every day at roughly the same time each day.

The terms “treat”, “treating”, and “treatment” refer to the eradication, removal, modification, management, or control of a tumor or primary, regional, or metastatic cancer cells or tissue and the minimization or delay of the spread of cancer.

The term “radiographic progression-free survival” or “rPFS” is defined as the occurrence of one of the following: (1) progression in bone, defined as the first bone scan with >2 new lesions compared with baseline is observed <12 weeks from randomization and is confirmed by a second bone scan taken ≥6 weeks later showing ≥2 additional new lesions (a total of ≥4 new lesions compared with baseline) or the first bone scan with ≥2 new lesions compared with baseline is observed ≥12 weeks from randomization and the ≥2 new lesions are verified on the next bone scan ≥6 weeks later (a total of ≥2 new lesions compared with baseline); (2) progression of soft tissue lesion measured by CT or MRI in modified RECIST 1.1 criteria; or (3) death.

The term “overall survival” or “OS” is defined as the time from randomization to the date of death from any cause.

The term “PFS2” means the time from initial study randomization to 2nd disease progression or death from any cause.

The term “time to PSA progression” is defined as the time from randomization to date of PSA progression based on Prostate Cancer Working Group 2 criteria. Scher H I, et al. J Clin Oncol 2008; 26:1148-1159.

The term “time to symptomatic progression” is defined as the easiest occurrent of a skeletal-related event, pain progression or worsening of disease-related symptoms requiring initiation of subsequent anti-cancer therapy, or development of clinically significant symptoms due to loco-regional tumor progression requiring surgical intervention or radiation.

The term “objective response rate” is defined per Response Evaluation Criteria in Solid Tumors (RECIST) v. 1.1.

The term “duration of response” is defined according to response criteria of Prostate Cancer Working Group 3 (PCWG3) for prostate cancer or RECIST v1.1.

The term “randomization” as it refers to a clinical trial refers to the time when the patient is confirmed eligible for the clinical trial and gets assigned to a treatment arm.

The terms “kit” and “article of manufacture” are used as synonyms.

The term “subject” and “patient” and “human” are used interchangeably.

The term “adverse event” or “AE” is any untoward medical occurrence in a clinical study participant administered a pharmaceutical (investigational or non-investigational) product. An AE does not necessarily have a causal relationship with the intervention. An AE can therefore be any unfavorable and unintended sign (including an abnormal finding), symptom, or disease temporally associated with the use of a medicinal (investigational or non-investigational) product, whether or not related to that medicinal (investigational or non-investigational) product. (Definition per International Council on Harmonisation [ICH]). This includes any occurrence that is new in onset or aggravated in severity or frequency from the baseline condition, or abnormal results of diagnostic procedures, including laboratory test abnormalities.

The term “serious adverse event” or “SAE” is based on ICH and EU Guidelines on Pharmacovigilance for Medicinal Products for Human Use is any untoward medical occurrence that at any dose: results in death, is life-threatening, requires inpatient hospitalization or prolongation of existing hospitalization, results in persistent or significant disability/incapacity, is a congenital anomaly/birth defect, is a suspected transmission of any infectious agent via a medicinal product, or is medically important.

The term “composite response rate” is defined as either 90% reduction in PSA level from baseline (PSA-90), or objective response in participants with measurable disease, or both at 12 weeks.

The term “PSA90 rate” or “PSA90” is defined as the proportion of participants with PSA decline >90% at 12 weeks from baseline.

The term “PSA50 rate” or “PSA50” is defined as the proportion of participants with PSA decline >50% at 12 weeks from baseline.

### Methods of Dosing and Treatment Regimens

In the following disclosure, “methods of treating metastatic castration-resistant prostate cancer,” may alternatively be recited as “methods of treating a male human having metastatic castration-resistant prostate cancer.” For the sake of brevity, each possible alternative is not parsed out, but each are considered separately considered as if fully described.

### The Compound of Formula I/Abiraterone Acetate-Corticosteroid Combination Therapy

Described herein are methods of treating mCRPC in a male human, said methods comprising, consisting of, or consisting essentially of, administering a therapeutically effective amount of the compound of formula I and abiraterone acetate and a corticosteroid to said male human.

In certain embodiments, the compound of formula I is administered daily to the male human. In further embodiments, the compound of formula I is administered orally to the male human. In some embodiments, the compound of formula I is administered orally to the male human on a continuous daily dosing schedule.

In some embodiments, the compound of formula I is administered orally to the male human at a dose of from about 600 mg per day to about 1000 mg per day, including all doses and subranges therebetween. In some embodiments, the compound of formula I is administered orally to the male human at a dose of about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, or about 1000 mg per day. In certain embodiments, the compound of formula I is administered orally to the male human at a dose of about 600 mg per day. In certain embodiments, the compound of formula I is administered orally to the male human at a dose of about 800 mg per day. In certain embodiments, the compound of formula I is administered orally to the male human at a dose of about 1000 mg per day.

In some embodiments, abiraterone acetate is administered at a dose of about 500 mg per day to about 1000 mg per day. In some embodiments, abiraterone acetate is administered at a dose of about 1000 mg per day.

In some embodiments, abiraterone acetate is administered in combination with a corticosteroid. In certain embodiments, the corticosteroid is prednisone, a prednisolone, or dexamethasone. In certain embodiments, the corticosteroid is prednisone. In certain embodiments, the corticosteroid is prednisolone. In certain embodiments, the corticosteroid is dexamethasone.

In further embodiments, the corticosteroid is administered at a dose of about 5 mg twice per day. In further embodiments, the prednisone is administered at a dose of about 5 mg twice per day. In further embodiments, the prednisolone is administered at a dose of about 5 mg twice per day.

In further embodiments, the dexamethasone is administered at a dose of about 0.5 mg to about 1.0 mg once daily, twice daily or three times daily. In further embodiments, the dexamethasone is administered at a dose of about 0.5 mg once daily. In further embodiments, the dexamethasone is administered at a dose of about 0.5 mg twice daily. In further embodiments, the dexamethasone is administered at a dose of about 0.5 mg three times daily. In further embodiments, the dexamethasone is administered at a dose of about 0.75 mg once daily. In further embodiments, the dexamethasone is administered at a dose of about 0.75 mg twice daily. In further embodiments, the dexamethasone is administered at a dose of about 0.75 mg three times daily. In further embodiments, the dexamethasone is administered at a dose of about 1.0 mg once daily. In further embodiments, the dexamethasone is administered at a dose of about 1.0 mg twice daily. In further embodiments, the dexamethasone is administered at a dose of about 1.0 mg three times daily.

In some embodiments, abiraterone acetate is administered once a day and the corticosteroid is administered once a day. In some embodiments, abiraterone acetate is administered once a day and the corticosteroid is administered twice a day. In some embodiments, abiraterone acetate is administered once a day and the corticosteroid is administered three times a day.

In certain embodiments the active ingredients, e.g., the compound of formula I and abiraterone acetate plus the corticosteroid, are administered to a patient as separate units or separate dosage forms, either simultaneously, concurrently or sequentially with no specific intervening time limits, wherein such administration provides safe and effective levels of the two active ingredients in the body of the human male. In certain embodiments, the compound of formula I may be administered prior to administration of abiraterone acetate plus the corticosteroid. In certain embodiments, the compound of formula I may be administered following administration of abiraterone acetate plus the corticosteroid.

In certain embodiments, prior to administration of the compound of formula I and abiraterone acetate plus the corticosteroid, the male human has been diagnosed with non-metastatic castration-resistant prostate cancer, metastatic castration-resistant prostate cancer, metastatic castration-sensitive prostate cancer, or locally advanced prostate cancer.

In some embodiments, administration of the compound of formula I and abiraterone acetate plus the corticosteroid provides a composite response rate in the male human. In some embodiments, administration of the compound of formula I and abiraterone acetate plus the corticosteroid provides an improved composite response rate in the male human.

Further described herein are methods of achieving a composite response rate in a male human with mCRPC, said methods comprising, consisting of, or consisting essentially of, administering a therapeutically effective amount of the compound of formula I and abiraterone acetate plus a corticosteroid to said male human.

Further described herein are methods of improving a composite response rate in a male human with mCRPC, said methods comprising, consisting of, or consisting essentially of, administering a therapeutically effective amount of the compound of formula I and abiraterone acetate plus a corticosteroid to said male human.

In any of the foregoing embodiments, the composite response rate comprises a PSA90, an objective response rate, or both. In certain embodiments, the composite response rate comprises a PSA90. In certain embodiments, the composite response rate comprises an objective response rate. In certain embodiments, the composite response rate comprises a PSA90 and an objective response rate. In certain embodiments, the composite response rate is at 12 weeks post administration.

In some embodiments, administration of the compound of formula I and abiraterone acetate plus a corticosteroid provides a PSA90 in the male human. In some embodiments, administration of the compound of formula I and abiraterone acetate plus a corticosteroid provides an improved PSA90 in the male human.

Further described herein are methods of improving PSA response rate in a male human with mCRPC, said methods comprising, consisting of, or consisting essentially of, administering a therapeutically effective amount of the compound of formula I and abiraterone acetate plus a corticosteroid to said male human. In certain embodiments, the PSA response rate is PSA50. In certain embodiments, the PSA response rate is PSA90.

In some embodiments, administration of the compound of formula I and abiraterone acetate plus the corticosteroid provides an objective response rate in the male human. In some embodiments, administration of the compound of formula I and the abiraterone acetate plus the corticosteroid provides an improved objective response rate in the male human.

Further described herein are methods of improving objective response rate in a male human with mCRPC, said methods comprising, consisting of, or consisting essentially of, administering a therapeutically effective amount of the compound of formula I and abiraterone acetate plus a corticosteroid to said male human.

In some embodiments, administration of the compound of formula I and the abiraterone acetate plus the corticosteroid provides an improvement in incidence and severity of adverse events in the male human. In certain embodiments, the adverse events comprise dose-limiting toxicities.

Further described herein are methods of improving incidence and severity of adverse events in a male human with mCRPC, said methods comprising, consisting of, or consisting essentially of, administering a therapeutically effective amount of the compound of formula I and abiraterone acetate plus a corticosteroid to said male human. In certain embodiments, the adverse events comprise dose-limiting toxicities.

In some embodiments, administration of the compound of formula I, abiraterone acetate plus a corticosteroid provides an improvement in radiographic progression-free survival (rPFS) in the male human.

Further described herein are methods of improving rPFS in a male human with mCRPC, said methods comprising, consisting of, or consisting essentially of, administering a therapeutically effective amount of the compound of formula I and abiraterone acetate plus a corticosteroid to said male human.

In any of the foregoing embodiments, the improvement may be relative to a population of male humans with mCRPC who did not receive an androgen receptor signaling inhibitor. In any of the foregoing embodiments, the improvement may be relative to a population of male humans with mCRPC who received docetaxel. In any of the foregoing embodiments, the improvement may be relative to a population of male humans with mCRPC who received a placebo. In any of the foregoing embodiments, the improvement may be relative to a population of male humans with mCRPC who received no treatment.

In some embodiments, the population to whom the compound of formula I and abiraterone acetate plus a corticosteroid is administered and the comparative population both have been previously been treated by the same or similar prior treatment regimen.

### The Compound of Formula I/Apalutamide Combination Therapy

Described herein are methods of treating mCRPC in a male human, said methods comprising, consisting of, or consisting essentially of, administering a therapeutically effective amount of the compound of formula I and a therapeutically effective amount of apalutamide to said male human.

In further embodiments, the compound of formula I is administered daily to the male human. In still further embodiments, the compound of formula I is administered orally to the male human. In some embodiments, the compound of formula I is administered orally to the male human on a continuous daily dosing schedule.

In some embodiments, the compound of formula I is administered orally to the male human at a dose of from about 600 mg per day to about 1000 mg per day, including all doses and subranges therebetween. In some embodiments, the compound of formula I is administered orally to the male human at a dose of about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, or about 1000 mg per day. In certain embodiments, the compound of formula I is administered orally to the male human at a dose of about 600 mg per day. In certain embodiments, the compound of formula I is administered orally to the male human at a dose of about 800 mg per day. In certain embodiments, the compound of formula I is administered orally to the male human at a dose of about 1000 mg per day.

In some embodiments, abiraterone acetate is administered at a dose of about 500 mg per day to about 1000 mg per day. In some embodiments, abiraterone acetate is administered at a dose of about 1000 mg per day.

In further embodiments, apalutamide is administered daily to the male human. In still further embodiments, apalutamide is administered orally to the male human. In some embodiments, apalutamide is administered orally to the male human on a continuous daily dosing schedule.

In some embodiments, apalutamide is administered orally to the male human at a dose of about 30 mg per day to about 480 mg per day. In further embodiments, apalutamide is administered orally to the male human at a dose of about 180 mg per day to about 480 mg per day. In certain embodiments, apalutamide is administered orally to the male human at a dose of: (a) about 30 mg per day; (b) about 60 mg per day; (c) about 90 mg per day; (d) about 120 mg per day; or (d) about 240 mg per day. In some embodiments, apalutamide is administered orally to the male human at a dose of about 240 mg per day. In certain embodiments, apalutamide is administered orally to the male human at a dose of about 60 mg and at a frequency of four times per day.

In certain embodiments the active agents, e.g., the compound of formula I and apalutamide, are administered to a patient as separate units or separate dosage forms, either simultaneously, concurrently or sequentially with no specific intervening time limits, wherein such administration provides safe and effective levels of the two active ingredients in the body of the human male. In certain embodiments, the compound of formula I may be administered prior to administration of apalutamide. In certain embodiments, the compound of formula I may be administered following administration of apalutamide.

In some embodiments, administration of the compound of formula I and apalutamide provides a composite response rate in the male human. In some embodiments, administration of the compound of formula I and apalutamide provides an improved composite response rate in the male human.

Further described herein are methods of achieving a composite response rate in a male human with mCRPC, said methods comprising, consisting of, or consisting essentially of, administering a therapeutically effective amount of the compound of formula I and apalutamide to said male human

Further described herein are methods of improving a composite response rate in a male human with mCRPC, said methods comprising, consisting of, or consisting essentially of, administering a therapeutically effective amount of the compound of formula I and apalutamide to said male human.

In any of the foregoing embodiments, the composite response rate comprises a PSA90, an objective response rate, or both. In certain embodiments, the composite response rate comprises a PSA90. In certain embodiments, the composite response rate comprises an objective response rate. In certain embodiments, the composite response rate comprises a PSA90 and an objective response rate. In certain embodiments, the composite response rate is at 12 weeks post administration.

In some embodiments, administration of the compound of formula I and apalutamide provides a PSA90 in the male human. In some embodiments, administration of the compound of formula I and apalutamide provides an improved PSA90 in the male human.

Further described herein are methods of improving PSA response rate in a male human with mCRPC, said methods comprising, consisting of, or consisting essentially of, administering a therapeutically effective amount of the compound of formula I and apalutamide to said male human. In certain embodiments, the PSA response rate is PSA50. In certain embodiments, the PSA response rate is PSA90.

In some embodiments, administration of the compound of formula I and apalutamide provides an objective response rate in the male human. In some embodiments, administration of the compound of formula I and apalutamide provides an improved objective response rate in the male human.

Further described herein are methods of improving objective response rate in a male human with mCRPC, said methods comprising, consisting of, or consisting essentially of, administering a therapeutically effective amount of the compound of formula I and apalutamide to said male human.

In some embodiments, administration of the compound of formula I and apalutamide provides an improvement in incidence and severity of adverse events in the male human. In certain embodiments, the adverse events comprise dose-limiting toxicities.

Further described herein are methods of improving incidence and severity of adverse events in a male human with mCRPC, said methods comprising, consisting of, or consisting essentially of, administering a therapeutically effective amount of the compound of formula I and apalutamide to said male human. In certain embodiments, the adverse events comprise dose-limiting toxicities.

In some embodiments, administration of the compound of formula I, apalutamide provides an improvement in radiographic progression-free survival (rPFS) in the male human.

Further described herein are methods of improving rPFS in a male human with mCRPC, said methods comprising, consisting of, or consisting essentially of, administering a therapeutically effective amount of the compound of formula I and apalutamide to said male human.

In any of the foregoing embodiments, the improvement may be relative to a population of male humans with mCRPC who did not receive an androgen receptor signaling inhibitor. In any of the foregoing embodiments, the improvement may be relative to a population of male humans with mCRPC who received docetaxel. In any of the foregoing embodiments, the improvement may be relative to a population of male humans with mCRPC who received a placebo. In any of the foregoing embodiments, the improvement may be relative to a population of male humans with mCRPC who received no treatment.

In certain embodiments, the androgen receptor signaling inhibitor is apalutamide.

In some embodiments, the population to whom the compound of formula I and apalutamide is administered and the comparative population both have been previously been treated by the same or similar prior treatment regimen.

In further embodiments, administration of the compound of formula I in combination with (a) abiraterone acetate plus a corticosteroid or (b) apalutamide, each (a) and (b) further in combination with androgen deprivation therapy. at least one GnRH agonist or antagonist. In some embodiments, the compound of formula I is administered in combination with abiraterone acetate plus a corticosteroid or apalutamide is further in combination with at least one GnRH agonist or antagonist. In some embodiments, the compound of formula I is administered in combination with abiraterone acetate, a corticosteroid, and at least one GnRH agonist or antagonist. In further embodiments, the compound of formula I is administered in combination with apalutamide and at least one GnRH agonist or antagonist.

In certain embodiments, administration of the compound of formula I in combination with abiraterone acetate plus a corticosteroid or apalutamide is further in combination with androgen deprivation therapy. In some embodiments, the compound of formula I is administered in combination with abiraterone acetate plus a corticosteroid, and androgen deprivation therapy. In further embodiments, the compound of formula I is administered in combination with apalutamide and androgen deprivation therapy.

In some embodiments, the androgen deprivation therapy consists of bilateral orchiectomy or gonadotropin-releasing hormone agonists or antagonists. In certain embodiments, castrated levels of testosterone are maintained by administering a GnRH agonist or antagonist or by orchiectomy.

In further embodiments, administration of the compound of formula I in combination with abiraterone acetate plus a corticosteroid or apalutamide is further in combination with at least one GnRH agonist or antagonist. In some embodiments, the compound of formula I is administered in combination with abiraterone acetate, a corticosteroid, and at least one GnRH agonist or antagonist. In further embodiments, the compound of formula I is administered in combination with apalutamide and at least one GnRH agonist or antagonist.

In still further embodiments, the at least one GnRH agonist or antagonist is or comprises leuprolide, buserelin, naferelin, histrelin, goserelin, deslorelin, degarelix, ozarelix, ABT-620 (elagolix), TAK-385 (relugolix), EP-100, KLH-2109 or triptorelin.

Physicians can prescribe GnRH agonists in accordance with instructions, recommendations and practices. In some embodiments, the gonadotropin-releasing hormone agonist or antagonist is leuprolide. In some embodiments, leuprolide is administered as a depot injection at a dose of about 7.5 mg every 4 weeks, or 22.5 mg every 3 months, or about 30 mg every 4 months, or about 45 mg every 6 months. In some embodiments, leuprolide is administered at about 0.01 mg to about 200 mg of leuprolide over a period of about 3 days to about 12 months, preferably about 3.6 mg of leuprolide over a period of about 3 days to about 12 months. In some embodiments, the gonadotropin-releasing hormone agonist or antagonist is buserelin. In some embodiments, the gonadotropin-releasing hormone agonist or antagonist is naferelin. In some embodiments, the gonadotropin-releasing hormone agonist or antagonist is histrelin. In some embodiments, the gonadotropin-releasing hormone agonist or antagonist is histrelin acetate. In some embodiments, histrelin acetate is administered at about 50 mg of histrelin acetate over a period of 12 months of histrelin acetate or about 50 μg per day of histrelin acetate. In some embodiments the GnRH agonist or antagonist is goserelin. In some embodiments, goserelin is administered as a subcutaneous implant at a dose of about 3.6 mg every 4 weeks or about 10.8 mg every 12 weeks. In some embodiments, goserelin is administered at about 0.01 mg to about 20 mg of goserelin over a period of about 28 days to about 3 months, preferably about 3.6 mg to about 10.8 mg of goserelin over a period of about 28 days to about 3 months. In some embodiments the GnRH agonist or antagonist is deslorelin. In some embodiments, the gonadotropin-releasing hormone agonist or antagonist is degarelix. In some embodiments, degarelix is administered as a subcutaneous injection at a dose of about 240 mg followed by about 80 mg administered every 4 weeks. In some embodiments the GnRH agonist or antagonist is ozarelix. In some embodiments the GnRH agonist or antagonist is ozarelix. In some embodiments the GnRH agonist or antagonist is ABT-620 (elagolix). In some embodiments the GnRH agonist or antagonist is TAK-385 (relugolix). In some embodiments the GnRH agonist or antagonist is EP-100. In some embodiments the GnRH agonist or antagonist is KLH-2109. In some embodiments, the gonadotropin-releasing hormone agonist or antagonist is triptorelin. In some embodiments, triptorelin is administered at about 0.01 mg to about 20 mg of triptorelin over a period of about 1 month, preferably about 3.75 mg of triptorelin over a period of 1 month.

In certain embodiments, administration of the compound of formula I in combination with abiraterone acetate plus a corticosteroid or apalutamide is further in combination with orchiectomy. In further embodiments, the orchiectomy is bilateral orchiectomy.

### Prior Therapy

In some embodiments, the male human has previously been administered docetaxel. In some embodiments, prior to administration of the compound of formula I and the abiraterone acetate plus a corticosteroid, the male human has previously been administered docetaxel. In some embodiments, prior to administration of the compound of formula I and apalutamide, the male human has previously been administered docetaxel. In certain embodiments, docetaxel has been administered intravenously at a dose of about 75 mg/m². In certain embodiments, docetaxel has been administered intravenously at a dose of about 75 mg/m² every 3 weeks for 6 cycles.

In some embodiments, the male human has not previously been administered apalutamide. In some embodiments, prior to administration of the compound of formula I and the abiraterone acetate plus a corticosteroid, the male human has not previously been administered apalutamide. In some embodiments, prior to administration of the compound of formula I and apalutamide, the male human has not been administered apalutamide.

### Routes of Administration and Pharmaceutical Compositions

Therapeutic agents described herein are administered in any suitable manner or suitable formulation.

Unless otherwise specified, the terms “effective amount” or “therapeutically effective amount,” as used herein, refer to an amount of an anti-androgen being administered that treats the underlying disease or condition including. halting or slowing the progression of the disease or condition.

The term “acceptable” with respect to a formulation, composition or ingredient, as used herein, means that the beneficial effects of that formulation, composition or ingredient on the general health of the male human being treated substantially outweigh its detrimental effects, to the extent any exist.

The compound of formula I, abiraterone acetate, corticosteroid, and apalutamide are each individually present in solid oral dosage forms. In some embodiments, they are formulated as an oral dose form, a unit oral dose form, or a solid dose form (e.g., a capsule, tablet, or pill). In some embodiments, for example, they are each formulated as a tablet.

To prepare the pharmaceutical compositions of this invention, the active pharmaceutical ingredient is intimately admixed with a pharmaceutical carrier according to conventional pharmaceutical compounding techniques, which carrier may take a wide variety of forms depending of the form of preparation desired for administration (e.g., oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

In solid oral preparations such as, for example, dry powders for reconstitution or inhalation, granules, capsules, caplets, gel caps, pills and tablets (each including immediate release, timed release and sustained release formulations), suitable carriers and additives include but are not limited to diluents, granulating agents, lubricants, binders, glidants, disintegrating agents and the like. Because of their ease of administration, tablets and capsules represent the most advantageous oral dosage unit form, in which case solid pharmaceutical carriers are obviously employed. If desired, tablets may be sugar coated, gelatin coated, film coated or enteric coated by standard techniques.

Preferably these compositions are in unit dosage forms from such as tablets, pills, capsules, dry powders for reconstitution or inhalation, granules, lozenges, sterile solutions or suspensions, metered aerosol or liquid sprays, drops, or suppositories for administration by oral, intranasal, sublingual, intraocular, transdermal, rectal, vaginal, dry powder inhaler or other inhalation or insufflation means.

These formulations are manufactured by conventional formulation techniques. For preparing solid pharmaceutical compositions such as tablets, the principal active ingredient is mixed with a pharmaceutical carrier, e.g., conventional tableting ingredients such as diluents, binders, adhesives, disintegrants, lubricants, antiadherents, and glidants. Suitable diluents include, but are not limited to, starch (i.e. corn, wheat, or potato starch, which may be hydrolized), lactose (granulated, spray dried or anhydrous), sucrose, sucrose-based diluents (confectioner's sugar; sucrose plus about 7 to 10 weight percent invert sugar; sucrose plus about 3 weight percent modified dextrins; sucrose plus invert sugar, about 4 weight percent invert sugar, about 0.1 to 0.2 weight percent cornstarch and magnesium stearate), dextrose, inositol, mannitol, sorbitol, microcrystalline cellulose (i.e. AVICEL microcrystalline cellulose available from FMC Corp.), dicalcium phosphate, calcium sulfate dihydrate, calcium lactate trihydrate and the like. Suitable binders and adhesives include, but are not limited to acacia gum, guar gum, tragacanth gum, sucrose, gelatin, glucose, starch, and cellulosics (i.e. methylcellulose, sodium carboxymethylcellulose, ethylcellulose, hydroxypropylmethylcellulose, hydroxypropylcellulose, and the like), water soluble or dispersible binders (i.e. alginic acid and salts thereof, magnesium aluminum silicate, hydroxyethylcellulose [i.e. TYLOSE available from Hoechst Celanese], polyethylene glycol, polysaccharide acids, bentonites, polyvinylpyrrolidone, polymethacrylates and pregelatinized starch) and the like. Suitable disintegrants include, but are not limited to, starches (corn, potato, etc.), sodium starch glycolates, pregelatinized starches, clays (magnesium aluminum silicate), celluloses (such as crosslinked sodium carboxymethylcellulose and microcrystalline cellulose), alginates, pregelatinized starches (i.e. corn starch, etc.), gums (i.e. agar, guar, locust bean, karaya, pectin, and tragacanth gum), cross-linked polyvinylpyrrolidone and the like. Suitable lubricants and antiadherents include, but are not limited to, stearates (magnesium, calcium and sodium), stearic acid, talc waxes, stearowet, boric acid, sodium chloride, DL-leucine, carbowax 4000, carbowax 6000, sodium oleate, sodium benzoate, sodium acetate, sodium lauryl sulfate, magnesium lauryl sulfate and the like. Suitable glidants include, but are not limited to, talc, cornstarch, silica (i.e. CAB-O-SIL silica available from Cabot, SYLOID silica available from W.R. Grace/Davison, and AEROSIL silica available from Degussa) and the like. Sweeteners and flavorants may be added to chewable solid dosage forms to improve the palatability of the oral dosage form. Additionally, colorants and coatings may be added or applied to the solid dosage form for ease of identification of the drug or for aesthetic purposes. These carriers are formulated with the pharmaceutical active to provide an accurate, appropriate dose of the pharmaceutical active with a therapeutic release profile.

Binders suitable for use in the pharmaceutical compositions provided herein include, but are not limited to, starches, cellulose, and its derivatives (e.g., ethylcellulose, cellulose acetate, carboxymethyl cellulose calcium, sodium carboxymethyl cellulose, methylcellulose, hydroxypropyl methylcellulose), polyvinyl pyrrolidone, and mixtures thereof.

Examples of fillers suitable for use in the pharmaceutical compositions provided herein include, but are not limited to, microcrystalline cellulose, powdered cellulose, mannitol, lactose, calcium phosphate, starch, pre-gelatinized starch, and mixtures thereof.

The binder or filler in pharmaceutical compositions is typically present in from about 50 to about 99 weight percent of the pharmaceutical composition or dosage form.

Disintegrants can be used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant. Disintegrants that can be used in the pharmaceutical compositions provided herein include, but are not limited to, croscarmellose sodium, crospovidone, sodium starch glycolate, potato or tapioca starch, pre-gelatinized starch, other starches, other celluloses, gums, and mixtures thereof.

Lubricants that can be used in the pharmaceutical compositions provided herein include, but are not limited to, calcium stearate, magnesium stearate, mineral oil, light mineral oil, glycerin, sorbitol, polyethylene glycol, other glycols, stearic acid, sodium lauryl sulfate, sodium stearyl fumarate, talc, hydrogenated vegetable oil (e.g., peanut oil, cottonseed oil, sunflower oil, sesame oil, olive oil, com oil, and soybean oil), zinc stearate, ethyl oleate, ethyl laureate, agar, and mixtures thereof. Lubricants are typically used in an amount of less than about 1 weight percent of the pharmaceutical compositions or dosage forms into which they are incorporated.

Compressed tablet formulations may optionally be film-coated to provide color, light protection, and/or taste-masking. Tablets may also be coated so as to modulate the onset, and/or rate of release in the gastrointestinal tract, so as to optimize or maximize the biological exposure of the patient to the API.

Hard capsule formulations may be produced by filling a blend or granulation of the compound of formula I into shells consisting of, for example, gelatin, or hypromellose.

Soft gel capsule formulations may be produced.

Pharmaceutical compositions intended for oral use may be prepared from the solid dispersion formulations, and blended materials described above in accordance with the methods described herein, and other methods known to the art for the manufacture of pharmaceutical compositions. Such compositions may further contain one or more agents selected from the group consisting of sweetening agents, flavoring agents, coloring agents, and preserving agents in order to provide pharmaceutically elegant and palatable preparations.

Tablets may contain the active ingredient in admixture with non-toxic pharmaceutically acceptable excipients that are suitable for the manufacture of tablets. These excipients may be for example, inert diluents, granulating, and disintegrating agents, binding agents, glidants, lubricating agents, and antioxidants, for example, propyl gallate, butylated hydroxyanisole, and butylated hydroxy toluene. The tablets may be uncoated, or they may be film coated to modify their appearance or may be coated with a functional coat to delay disintegration, and absorption in the gastrointestinal tract, and thereby provide a sustained action over a longer period.

Compositions for oral use may also be presented as capsules (e.g., hard gelatin) wherein the active ingredient is mixed with an inert solid diluent, for example, calcium carbonate, calcium phosphate or starch, or as soft gelatin capsules wherein the active ingredient is mixed with liquids or semisolids, for example, peanut oil, liquid paraffin, fractionated glycerides, surfactants or olive oil. Aqueous suspensions contain the active materials in mixture with excipients suitable for the manufacture of aqueous suspensions. Dispersible powders and granules suitable for preparation of an aqueous suspension by the addition of water provide the active ingredient in mixture with a dispersing or wetting agent, suspending agent, and one or more preservatives. In certain embodiments of the invention, the pharmaceutical compositions of the invention include a diluent system, disintegrant, salt, lubricant, glidant, and filmcoat, at concentrations of from about 3% w/w to about 58% w/w, from about 4% w/w to about 20% w/w, from about 4% w/w to about 20% w/w, from about 0.5% w/w to about 4% w/w, from about 0% w/w to about 2% w/w, and from about 1% w/w to about 5% w/w respectively, or at from about 18% w/w to about 40% w/w, from about 7% w/w to about 15% w/w, from about 7% w/w to about 18% w/w, from about 1.0% w/w to about 3.0%, from about 0.1% w/w to about 1.0% w/w, and from about 2.0% w/w to about 4.0% w/w, respectively. In certain embodiments, the solid dispersion formulations are blended with a diluent, one or more disintegrating agents, lubricants, and glidants. An exemplary blended composition or oral dosage form includes mannitol, microcrystalline cellulose, croscarmellose sodium, sodium chloride, colloidal silica, sodium stearyl fumarate, and magnesium stearate.

The disintegrant may be present in a concentration from about 4% w/w to about 20% w/w or from about 7% w/w to about 15% w/w. A salt may be also present, which may be sodium chloride, potassium chloride or a combination thereof. The combination of salts and disintegrant is present at a concentration from about 5% w/w to about 35% w/w of the final pharmaceutical composition.

In certain embodiments, inactive ingredients of the core tablet are: colloidal anhydrous silica, croscarmellose sodium, hydroxypropyl methylcellulose-acetate succinate, magnesium stearate, microcrystalline cellulose, and silicified microcrystalline cellulose. In other embodiments, the tablets are finished with a film-coating consisting of the following excipients: iron oxide black, iron oxide yellow, polyethylene glycol, polyvinyl alcohol, talc, and titanium dioxide

In other embodiments, a single unit dosage of the pharmaceutical composition comprises, consists of, or consists essentially of about 200 mg of the compound of formula I. The total daily dose of the compound of formula I may be about 600 mg per day. The total daily dose of the compound of formula I may be about 800 mg per day. The total daily dose of the compound of formula I may be about 1000 mg per day.

All formulations for oral administration are in dosage form suitable for such administration.

### Kits/Articles of Manufacture

For use in the therapeutic methods of use described herein, kits and articles of manufacture are also described herein. Such kits include a package or container that is compartmentalized to receive one or more dosages of the pharmaceutical compositions disclosed herein. Suitable containers include, for example, bottles. In one embodiment, the containers are formed from a variety of materials such as glass or plastic.

The articles of manufacture provided herein contain packaging materials. Packaging materials for use in packaging pharmaceutical products include, e.g., U.S. Pat. Nos. 5,323,907, 5,052,558 and 5,033,252. Examples of pharmaceutical packaging materials include, but are not limited to, blister packs, bottles, tubes, bags, containers, bottles, and any packaging material suitable for a selected formulation and intended mode of administration and treatment.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### EXAMPLES

The following example is provided for illustrative purposes only and not to limit the scope of the claims provided herein.

### EXAMPLE 1: A Phase 1b Study of the Compound of Formula I in Combination with (a) Abiraterone Acetate Plus Prednisone or Prednisolone or (b) Apalutamide in mCRPC

A non-limiting example of a Phase 1b Study the compound of formula I in combination with abiraterone acetate plus prednisone or prednisolone or apalutamide in metastatic castration-resistant prostate cancer is provided herein.

### Objectives and Endpoints

The primary and secondary objectives and endpoints are provided in Table 1.

TABLE 1

| | |
|---|---|
| Objectives | Endpoints |

Primary

| | |
|---|---|
| To determine safety, including dose-limiting | Incidence and severity of AEs, |
| toxicities, and the recommended Phase 2 dose | including dose-limiting toxicities |
| (RP2D) of the compound of formula I in separate | Composite response rate at 12 |
| combinations with (a) abiraterone acetate plus | weeks comprised of: |
| prednisone or prednisolone (AAP) and (b) | PSA90 rate, defined as the proportion |
| apalutamide (dose-finding) | of participants with PSA decline >90% |
| To determine the antitumor activity of the | at 12 weeks from baseline |
| compound of formula I in separate combinations | Objective response rate at 12 weeks |
| with (a) AAP and (b) apalutamide (dose-expansion) | in participants with measurable |
| | disease, per RECIST 1.1 |

Secondary

| | |
|---|---|
| To assess the pharmacokinetics following multiple | Pharmacokinetic parameters for the |
| doses | compound of formula I and |
| | abiraterone (Group A) including but |
| | not limited to Cₘₐₓ, Tₘₐₓ, AUCₜₐᵤ, |
| | Cₘᵢₙ, and accumulation ratio |
| | (AR_{Cmax} and AR_{AUCtau}) |
| | Pharmacokinetic parameters for the |
| | compound of formula I and |
| | apalutamide (Group B) including |
| | but not limited to Cₘₐₓ, Tₘₐₓ, |
| | AUCₜₐᵤ, Cₘᵢₙ, and accumulation |
| | ratio (AR_{Cmax} and AR_{AUCtau}) |
| To assess the pharmacodynamics following | Pharmacodynamic markers |
| multiple doses | including but not limited to serum |
| | prostate-specific antigen (PSA) |

Exploratory
To explore the relationships between pharmacokinetics, pharmacodynamics,
adverse event profile, and clinical activity of the compound of formula
I with either (A) AAP and (B) apalutamide
To investigate biomarkers predictive of clinical response or resistance
to the compound of formula I with either (A) AAP or (B) apalutamide
To assess radiographic progression-free survival (rPFS) of the compound
of formula I in combination with (A) AAP and (B) apalutamide

### Study Design

The study described in this example is a Phase 1b, open-label, multicenter study to evaluate the safety, pharmacokinetics, pharmacodynamics and antitumor activity of the compound of formula I in combination with AAP (Group A) or apalutamide (Group B) in participants with mCRPC with no prior second-generation ARSI treatment. The Study Evaluation Team (SET) continuously monitors all safety data, including DLTs and late-occurring toxicities. SET decisions (including dose escalation, determination of the RP2D, and dose expansion) are based on review of all available data, including pharmacokinetics, pharmacodynamics, safety, and antitumor activity.

This study enrolls adult mCRPC patients who have not received prior therapy with second-generation ARSIs for mCSPC, nmCRPC, or mCRPC. Participants may have received prior docetaxel for mCSPC with a maximum duration of 6 cycles (but must not have had disease progression during, or within 6 months of completing chemotherapy).

Approximately 18 evaluable participants are enrolled to the dose-finding portion of the study for each Group (A and B). In the dose expansion portion of the study, depending on efficacy detected in the dose-finding portion, up to 23 additional participants are enrolled for each Group. Participants continue on study treatment until death, radiographic progression, or unequivocal clinical progression, whichever occurs first.

### Study Drugs

Descriptions and administration instructions for study drugs are provided in Table 2.

TABLE 2

| | The compound of | | |
|---|---|---|---|
| | formula I | AAP | Apalutamide |
| Dosage formulation | 200 mg tablets | AA: 250 mg tablets; | 60 mg tablets |
| | | P: all commercial | |
| | | presentations are | |
| | | allowed | |
| Route of administration | Oral | Oral | Oral |
| Dose level/frequency | 800 mg QD (DL1) | AA: 1000 mg QD; | 240 mg QD; Refer to |
| | | P: 5 mg BID; | local product label for |
| | | Refer to local product | instructions on handling |
| | | label for instructions on | of missed doses |
| | | handling of missed | |
| | | doses | |
| Dosing instructions | There are no fasting | AA: QD on empty | Refer to local product |
| | restrictions with the | stomach. AA should be | label for instructions. |
| | compound of formula I. | taken at least one hour | There are no fasting |
| | | before or at least two | restrictions with |
| | | hours after eating. These | apalutamide |
| | | should be swallowed | |
| | | whole with water. | |
| | | P: recommended to be | |
| | | taken with food, twice | |
| | | daily (morning and | |
| | | evening) | |

| | |
|---|---|
| Missed doses | If a dose is missed and 6 or more hours have elapsed since the scheduled time of |
| | the administration, the dose should be skipped and the participant should continue |
| | treatment with the study drug at the scheduled time the next day. If a dose is |
| | missed by less than 6 hours, the participant should take the study drug immediately |
| | and resume treatment with the study drug at the scheduled time the next day. |
| | Missed doses for any reasons will not be allowed to be made up either by combining |
| | with scheduled daily dose or in the specified rest period. If vomiting occurs, the |
| | participant should be instructed that no replacement dose should be taken |

Abbreviations:
AA = abiraterone acetate;
AAP = abiraterone acetate plus prednisone or prednisolone;
BID = twice daily;
P = prednisone or prednisolone;
QD = once daily

### Dose Finding and Determination of the RP2D

Participants in the study must not have received second-generation ARSI treatment prior to enrollment. Participants may have had prior treatment with docetaxel for mCSPC with a maximum duration of 6 cycles (but must not have had disease progression during chemotherapy, or within 6 months of completing chemotherapy). Eligible participants are assigned sequentially to Group A or Group B. However, the sponsor may assign a participant to the alternative Group as clinically appropriate, based on the participant's baseline medical history and risk factors.

Dose levels for the compound of formula I in Part 1 are summarized in Table 3.

TABLE 3
Dose Levels of the compound of formula I for Groups A and B

| | Group A* | Group B* |
|---|---|---|
| Dose Level (DL) | AA 1000 mg PO QD + | Apalutamide |
| the compound of | P 5 mg PO BID, and the | 240 mg PO QD and the |
| formula I | compound of formula I: | compound of formula I: |
| DL2 | 1000 mg PO QD | 1000 mg PO QD |
| DL1 | 800 mg PO QD | 800 mg PO QD |
| DL-1^{#} | 600 mg PO QD | 600 mg PO QD |

BID = twice daily;
DL = dose level;
PO = by mouth;
QD = once daily
*In Group A, the dose of AAP is AA 1000 mg PO QD, prednisone or prednisolone 5 mg PO BID at all DLs. In Group B, the dose of apalutamide is 240 mg PO QD at all DLs. The Safety Evaluation Team (SET), after evaluation of safety/pharmacokinetic and other data, may determine an alternative dosing regimen for AAP or apalutamide.
^{#}The SET, after evaluating all safety and PK data emerging from DL1, may decide to open DL-1 even if DLT/treatment delay criteria are not met.

The first evaluable participant is accrued to DL1 for each Group. The dose-limiting toxicity (DLT) evaluation period is defined as the first 28 days (1 cycle) of combination treatment and is described in Section 4.1.3.
To determine the RP2D dose of the compound of formula I in combination with AAP or apalutamide, the guidelines described below are followed: DLTs are assessed by the SET based on the safety, pharmacokinetics, and other available data.
Dose escalation/de-escalation are based on Bayesian Optimal Interval (BOIN) Design. Participants are enrolled and treated in flexible cohort sizes based on SET decision. The safety data is monitored continuously. The target DLT rate is 28% and the planned maximum number of participants in either Group A or B is 18. The BOIN design uses the following rule, optimized to minimize the probability of incorrect dose assignment, to guide dose escalation/de-escalation:
   if the observed DLT rate at the current dose is ≤0.221, escalate the dose to the next higher dose level;
   if the observed DLT rate at the current dose is >0.334, de-escalate the dose to the next lower dose level;
   otherwise, if the observed DLT rate is >0.221 and ≤0.334, stay at the current dose.
The initial dose of the compound of formula I is 800 mg by mouth daily (1 dose level below the monotherapy RP2D); two other dose levels are considered: 1000 mg and 600 mg.
A strategy of staggered dosing with the compound of formula I is applied between the first and second participant in each cohort.
   For the first dose escalation cohort: there must be an observation period of at least 48 hours between administration of the compound of formula I in combination with AAP or apalutamide to the first and second participant. For subsequent cohorts the duration of the observation period may be modified by the SET based on emerging data.
   The SET evaluation of a cohort is flexible regarding the number of participants completing the DLT evaluation period at each dose level but will not exceed 6. This is based on SET agreement by monitoring the data. Before determining the dose for the next cohort, SET evaluation of the current cohort data is required.
The sponsor may decide to terminate either Group at any time due to safety or pharmacokinetic considerations.
The dose escalation/de-escalation is repeated until the maximum sample size of 18 is reached or the study is stopped if the number of evaluable participants treated at the current dose reaches 12 and the decision is to stay at the current dose.

The planned minimum number of participants in either Group A or B is 6 and the planned maximum number is 18If the SET opens a higher dose level, participants in the lower dose levels are escalated into the higher dose level if they have passed the DLT period.

### Determination of the RP2D

The RP2D is determined after review of all available pharmacokinetic, pharmacodynamic, safety, and efficacy data. The toxicity probability at each dose level is estimated based on isotonic estimates and the selection of the MTD is guided as the highest dose level, for which the isotonic estimate of the DLT rate is below the target rate of 28%, with consideration of all available PK, pharmacodynamic, safety, and efficacy data. If an MTD is determined, the RP2D regimen(s) may be lower than the MTD. If the MTD cannot be determined based on lack of toxicity, maximum administered dose (MAD) may be defined (Table 3).

### Definition of Dose-Limiting Toxicity (DLT)

The DLT evaluation period is defined as the first 28 days (1 cycle) of combination treatment. DLTs are assessed by the SET, along with safety, pharmacokinetic, and other available data. A DLT is defined as one of the following toxicities occurring in the DLT evaluation period (the SET may assess a toxicity that occurs outside the DLT evaluation period as a DLT, on a case-by-case basis), and is related to the compound of formula I, AAP, or apalutamide as assessed by the investigator and/or sponsor:
1. Grade 3 or higher non-hematologic adverse event, excluding the following:
   a. Grade 3 nausea, vomiting, fatigue, or diarrhea that resolves to Grade 1 or less within 7 days
   b. Grade 3 laboratory abnormality that deemed not clinically significant by the investigator and participant is asymptomatic
2. Hepatic-related adverse events:
   a. Grade 3 or higher elevation of transaminases (AST/ALT) OR of total bilirubin lasting >48 hours
   b. A possible Hy's law case, defined by the occurrence of ALT/AST ≥3×ULN, ALP <2×ULN together with bilirubin ≥2×ULN or INR >1.5 (if measured), may be considered as a DLT, on a case-by-case basis. All possible cases of Hy's Law must be reported to the sponsor immediately
3. Rash:
   a. In all participants, any Grade 4 rash or occurrence of toxic epidermal necrolysis (TEN)/Steven-Johnson syndrome (SJS) are considered a DLT
4. b. In participants enrolled to Group B (apalutamide+the compound of formula I), Grade 1-3 maculopapular rash will not be considered a DLT, as this is a known toxicity of apalutamide. In the dose-finding portion of the study, participants requiring treatment interruption of more than 14 days in the DLT period may be replaced at the sponsor's discretion Grade 3 or higher febrile neutropenia, or Grade 4 or higher neutropenia
5. Grade 4 or higher anemia or thrombocytopenia
6. Grade 3 or higher hyperglycemia lasting more than 7 days
7. Grade 4 or higher hypertension, or BP >160 mm Hg systolic and/or >100 mg diastolic for more than 7 days which cannot be controlled with medications
8. Any other toxicity, which, in the view of the investigator or the SET, is considered to be a DLT; these are discussed on a case-by-case basis by the SET.

### Number of Participants

Approximately 18 evaluable participants are enrolled to the dose-finding portion of the study for each Group (A and B). In the dose expansion portion of the study, depending on efficacy detected in the dose-finding portion, up to 23 additional participants will be enrolled for each Group. Participants who are non-evaluable for futility analysis will be replaced. The total planned number of participants in this study is approximately 41 for each Group

### Treatment Groups and Duration

Participants continue to receive study drug until radiographic disease progression, death, unequivocal clinical progression, unacceptable toxicity, withdrawal of consent, or lost to follow-up, whichever comes first. The end of study (study completion) is defined as the last assessment for the last participant on study.

### Study Population

Screening for eligible participants is performed within 30 days before administration of the study treatment.

The inclusion and exclusion criteria for enrolling participants in this study are described below.

### Inclusion Criteria

Each potential participant must satisfy all of the following criteria to be enrolled in the study:

### Age

1. 18 years of age or older.

### Type of Participant and Disease Characteristics

2. Histologically confirmed prostate adenocarcinoma
3 Progression of metastatic prostate cancer in the setting of castrate levels of testosterone ≤50 ng/dL on a gonadotropin releasing hormone analog (GnRHa), or history of bilateral orchiectomy at study entry defined as having one or more of the following:
   b. PSA progression defined by a minimum of 2 successive rising PSA levels with an interval of ≥1 week between each value. The PSA level at the screening visit must be ≥1 μg/L (1 ng/ml)
   c. Bone progression (+/−nodal disease), per PCWG3 criteria
   d. Extra-pelvic nodal progression (per modified RECIST 1.1 criteria)
   e. Visceral progression (per RECIST 1.1 criteria)
4. Prior systemic therapy for prostate cancer, as follows:
   a. No prior ARSIs for mCSPC, nmCRPC, or mCRPC. Prior bicalutamide, flutamide, or nilutamide is allowed when used temporarily (eg, for flare protection or combined with radiation therapy) in the localized, locally advanced, biochemical recurrence or mCSPC setting; any use of these agents after progression to mCRPC renders the participant ineligible.
   b. Prior docetaxel for mCSPC, up to maximum of 6 cycles is allowed, but participants must not have progressed during, or within 6 months of completing chemotherapy
5. Must be able to continue GnRHa during the study if not surgically castrate.
6. Eastern Cooperative Oncology Group (ECOG) performance status grade of 0, 1, or 2.
7. Hematology laboratory parameters within the following ranges, independent of transfusion within 7 days prior to first dose of study drug or growth factors within 3 weeks prior to first dose of study drug:
   f. Hemoglobin ≥9 g/dL
   g. Absolute neutrophil count ≥1.0×10⁹/L
   h. Platelets count ≥50×10⁹/L
8. Chemistry laboratory parameters within the following range before first administration of study drug:
   i. Estimated glomerular filtration rate (eGFR): ≥50 mL/min based upon Modified Diet in Renal Disease formula calculation (see Section Error! Reference source not found.; Appendix 9)
   j. Serum total bilirubin≤1.5× the upper limit of normal (ULN); in participants with congenital bilirubinemia, such as Gilbert's syndrome, if total bilirubin is >1.5×ULN but direct bilirubin is ≤1.5×ULN, participant is eligible
   k. Aspartate aminotransferase (AST) and alanine aminotransferase (ALT)≤2.5×ULN
   l. Potassium ≥3.5 mmol/L
   m. Chromogranin A<1.5× ULN
9. Must be able to swallow oral medicines.

### Sex and Contraceptive/Barrier Requirements

10. Must agree to all the following during the study and for 90 days after the last dose of study drug:
   n. Wear a condom when engaging in any activity that allows for passage of ejaculate to another person.
   o. Not to donate sperm or freeze for future use for the purpose of reproduction.
   p. Not plan to father a child
11. Contraceptive use by men (and female partners of men enrolled in the study who are of childbearing potential or are pregnant) (birth control) use should be consistent with local regulations regarding the use of contraceptive methods for participants participating in clinical studies.

### Informed Consent

12. Willing and able to adhere to the prohibitions and restrictions specified in this protocol.
13. Must sign an informed consent form (ICF) indicating that he understands the purpose of, and procedures required for, the study and is willing to participate in the study.

### Exclusion Criteria

Any potential participant who meets any of the following criteria is excluded from participating in the study:

### Medical Conditions

1. Known central nervous system (CNS) metastases.
2. Non-metastatic CRPC (biochemical or locoregional disease only) is excluded from trial participation.
3 Evidence of predominant neuroendocrine/small cell carcinoma features in archival or baseline tumor biopsy specimen(s).
4. Symptomatic or impending spinal cord compression, except if participant has received definitive treatment and demonstrates evidence of clinically stable disease.
5. Known disorder affecting gastrointestinal absorption.

### Prior/Concomitant Therapy

6. Toxicities from previous anticancer therapy, including radiation, must have resolved to baseline levels or to Grade 1 or less except for alopecia, vitiligo, radiation fibrosis, and peripheral neuropathy, which may be Grade 2. Participants with endocrinopathy who are stable on hormone replacement are permitted.

### Prior/Concurrent Medical Conditions

7. Recipients of solid organ or bone marrow transplantation within 5 years from randomization. If transplantation is longer than 5 years ago, enrollment is possible if there is no evidence of transplant rejection.
8. Active malignancies (i.e, progressing or requiring treatment change in the last 24 months) other than the disease being treated under study. The only allowed exceptions are:
   q. Non-muscle invasive bladder cancer (NMIBC).
   r. Skin cancer (non-melanoma or melanoma) treated within the last 24 months that is considered completely cured.
   s. Breast cancer:
      adequately treated lobular carcinoma in situ or ductal carcinoma in situ,
      or history of localized breast cancer and considered to have a very low risk of recurrence.
   t. Other malignancy that is considered at minimal risk of recurrence
9. Any of the following within 6 months prior to screening:
   u. Myocardial infarction
   v. Severe or unstable angina
   w. Clinically significant ventricular arrhythmias
   x. Congestive heart failure (New York Heart Association class II to IV)
   y. Cerebrovascular accident or transient ischemic attack (within 12 months prior to screening)
   Z. Any grade arterial thrombotic event
10. Uncontrolled hypertension (systolic blood pressure ≥160 mmHg or diastolic blood pressure ≥100 mmHg)
11. Venous thromboembolic events (i.e. pulmonary embolism) within 6 months prior to the first dose of study drug; uncomplicated (Grade≤2) deep vein thrombosis is not considered exclusionary.
12. Known allergies, hypersensitivity, or intolerance to the excipients of the compound of formula I, apalutamide or AAP.
13. Concurrent use of any other anticancer treatment or investigational agent for the treatment of advanced prostate cancer.
14. Active infection or condition that requires treatment with systemic antibiotics within 7 days prior to the first dose of study drug.
15. Participants who have undergone major surgery must have recovered adequately without sequelae at least 3 weeks prior to starting the study drug. Note: Participants with planned surgical procedures to be conducted under local anesthesia may participate.
16. Active hepatitis B or C infection according to local laboratory range, on all available tests for the past 6 months or other clinically active liver disease
   Seropositive for hepatitis B: defined by a positive test for hepatitis B surface antigen [HBsAg]. Participants with resolved infection (ie, participants who are HBsAg negative with antibodies to total hepatitis B core antigen [anti-HBc] with or without the presence of hepatitis B surface antibody [anti-HBs]) must be screened using real-time polymerase chain reaction (RT-PCR) measurement of hepatitis B virus (HBV) DNA levels. Those who are RT-PCR positive are excluded. Participants with serologic findings suggestive of HBV vaccination (anti-HBs positivity as the only serologic marker) AND a known history of prior HBV vaccination, do not need to be tested for HBV DNA by RT-PCR.
   Known hepatitis C infection or positive serologic testing for Hepatitis C (anti-HCV antibody.
   Positive hepatitis C antibody test result at screening or within 3 months prior to starting study treatment. NOTE: Participants with positive hepatitis C antibody due to prior resolved disease can be enrolled only if a confirmatory negative hepatitis C RNA test is obtained.
   Positive hepatitis C RNA test result at screening or within 3 months prior to first dose of study treatment. NOTE: Test is optional and participants with negative hepatitis C antibody test are not required to also undergo hepatitis C RNA testing.
17. Severe hepatic impairment Class C per Child-Pugh classification system
18. Any condition for which, in the opinion of the investigator or sponsor, participation would not be in the best interest of the participant or that could prevent, limit, or confound the protocol-specified assessments.
19. For those enrolling into Group B (the compound of formula I+apalutamide):
   aa. Any prior history of seizure
   bb. Loss of consciousness≤1 year prior to randomization
   cc. Presence of brain arteriovenous malformation
   dd. Intracranial masses such as schwannomas and meningiomas that are causing edema or mass effect
   ee. Ongoing treatment with drugs known to lower the seizure threshold or known to cause seizures
   ff. Any condition that may predispose to seizure (consult sponsor for review) Participants with the above conditions may be eligible to participate in this study in Group A, after review by sponsor
20. Participant is known to be positive for human immunodeficiency virus (HIV) with 1 or more of the following:
   Not receiving highly active antiretroviral therapy (ART).
   Had a change in antiretroviral therapy within 6 months of the start of screening
   Receiving antiretroviral therapy that may interfere with study treatment (consult Sponsor for review of medication prior to enrollment)
   CD4+ count <350 at screening
   AIDS-defining opportunistic infection within 6 months of start of screening.
   Does not agree to start ART and be on ART for >4 weeks plus having HIV viral load <400 copies/mL at the end of the 4-week period (to ensure ART is tolerated and HIV is controlled).

### Efficacy Evaluations

Composite response rate is used for the primary efficacy measure analysis. Other evaluations include investigator-assessed tumor measurements (i.e., chest, abdomen, and pelvis computed tomography [CT] or magnetic resonance imaging [MRI] scans and whole-body bone scans [99mTc]) and survival status.

### Pharmacokinetic, Pharmacodynamic, and Biomarker Evaluations

Blood samples are collected to measure the plasma concentration of the compound of formula I and, if warranted, its metabolites, when dosed with the combination agent. Systemic (serum or plasma) concentrations of each combination agent, and population PK parameters and derived exposure are determined.

Changes in PSA are evaluated as pharmacodynamic markers of AR inhibition. Additional exploratory pharmacodynamic biomarkers may be evaluated in whole blood or plasma.

Biomarker samples are collected and, if deemed to be of scientific value, are evaluated to understand the mechanism of action of the compound of formula I in combination with A) AAP and B) apalutamide or may help to identify subgroups that respond differently to the study drug combinations. Samples collected for biomarker evaluations include archival tumor specimens, whole blood, plasma, serum, and tumor biopsies at baseline and at progression, if available.

### Safety Evaluations

Safety assessments are based on medical review of AE reports and the results of vital sign measurements, physical examinations, clinical safety laboratory tests, electrocardiograms (ECG), Eastern Cooperative Oncology Group Performance Score, and other safety evaluations at specified timepoints.

### Statistical Hypothesis

The BOIN method will determine the RP2D for the compound of formula I in separate combinations with (A) AAP and (B) apalutamide with an upper bound for the true DLT rate equal to 28%.

The sponsor will monitor the futility of each combination separately by applying Simon's 2-stage which will test for the null hypothesis of composite response rate of <48% against composite response rate >70%.

### Statistical Methods

Statistical analysis are performed for each treatment combination separately. Bayesian Optimal Interval (BOIN) design is applied to find the RP2D of the compound of formula I in separate combinations with AAP and apalutamide. Simon's 2-stage design is utilized to evaluate the futility of each combination. In the first stage, data from participants treated at the RP2D in the dose-finding portion of the study are used to evaluate futility. Data is summarized using descriptive statistics. Continuous variables are summarized using the number of observations, mean, standard deviation (SD), median, and range. Categorical values are summarized using the number of observations and percentages as appropriate.

### Dose Modification

In general, dose interruptions/modifications should be managed as described in Table 4:

TABLE 4
Dose Modifications for Toxicity for Drug-related
AEs (except for rash associated with apalutamide)

| Toxicity | General guidance for all study drugs |
|---|---|
| Grade 1 or 2 | No change |
| ≥Grade 3 | Hold until returns to Grade 1 or baseline and resume |
| | at full dose, or dose reduction (at the investigator's |
| | discretion) |
| Recurrence ≥ | Hold until returns to Grade 1 or baseline, and |
| Grade 3 | resume with dose reduction. Up to 2 dose reductions |
| | are allowed for recurrent treatment-related toxicity. |
| | Discontinue if toxicity persists after 2 dose reductions. |
| | Discontinuation of study treatment should also be |
| | considered if toxicity does not resolve to Grade 1 or |
| | baseline within 2 cycles, after discussion with sponsor. |
| For Group B: | Discontinue apalutamide permanently |
| first occurrence | |
| of seizure of any | |
| grade or Grade | |
| 4 neurotoxicity | |

Note:
Adverse events are graded according to NCI-CTCAE Version 5.

The following general principles should be followed:
Grade 1 or Grade 2 toxicities should be managed symptomatically without dose adjustments; appropriate supportive medical treatment should be used.
In the event of a Grade 3 or higher toxicity which cannot definitively be attributed to the compound of formula I or AAP (Group A) or the compound of formula I or apalutamide (Group B), both drugs should be held until toxicity has resolved to Grade 1 or baseline
If Grade 3 or higher toxicity does not resolve to Grade 1 or baseline within 2 cycles, the participant should be discontinued from treatment or the investigator's rationale to continue treatment must be discussed with the sponsor.
Dose reduction guidelines and management of toxicities are provided below. A participant may have up to 2 dose adjustments for the same toxicity and if the same toxicity recurs at Grade 3 or higher after 2 dose adjustments, the participant should discontinue study treatment(s); any exceptions must be reviewed by the sponsor.
Dose modifications are provided as guidance and should not replace the investigator's own clinical judgment.

Dose escalation is permitted after resolution of toxicities to Grade≤1 or baseline. The investigator's rationale to re-escalate treatment must be discussed with the sponsor's medical monitor on an individual basis prior to implementation

### Drug-specific Guidance for Dose Modifications

### The Compound of Formula I

After the DLT period, at each dose level, up to 2 dose reductions are permitted (e.g., from 1000 mg to 800 mg; from 800 mg to 600 mg), based on review of emerging safety and pharmacokinetic data.

### AAP and Apalutamide

For abiraterone acetate, up to 2 dose-level reductions are permitted. At each dose-level reduction, the dose is reduced by 250 mg of AA: 1000 mg to 750 mg, or 750 mg to 500 mg. Doses below 500 mg are not permitted. Dosing of prednisone may be decreased from 5 mg BID to 5 mg once daily, at the investigator's discretion.

For apalutamide, up to 2 dose reductions are allowed. At each dose-level reduction, the dose is reduced by one tablet (60 mg) of apalutamide: 240 mg to 180 mg, or 180 mg to 120 mg. Doses below 120 mg apalutamide are not permitted.

If a participant has clinical progression—i.e. worsening ECOG PS to >3, use of opiates for ≥3 weeks, radiotherapy or surgery for the treatment of tumor progression-even if no radiographic disease progression is confirmed-study treatment can be stopped based on physician's discretion and subsequent treatment initiated.

Participants with clinical benefit as deemed by the investigator despite PSA and/or radiographic progression may continue treatment after consultation with the sponsor.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

The invention is further defined by the following numbered embodiments:

1. A method of treating metastatic castration-resistant prostate cancer (mCRPC) in a male human, said method comprising administering a therapeutically effective amount of a compound of formula I and a therapeutically effective amount of abiraterone acetate plus a therapeutically effective amount of a corticosteroid to said male human

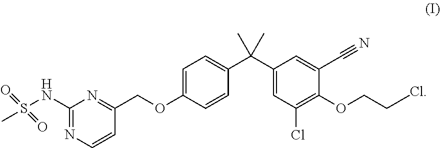

2. The method of embodiment 1, wherein the compound of formula I is administered orally to the male human at a dose of from about 600 mg per day to about 1000 mg per day.
3. The method of embodiment 1 or 2, wherein the compound of formula I is administered orally to the male human at a dose of:
(a) about 600 mg per day;
(b) about 800 mg per day; or
(c) about 1000 mg per day.
   4. The method of any one of the preceding embodiments, wherein the compound of formula I is administered orally to the male human at a dose of about 800 mg per day.
   5. The method of any one of the preceding embodiments, wherein abiraterone acetate is administered orally to the male human at a dose of about 500 mg per day to about 1000 mg per day.
   6. The method of any one of the preceding embodiments, wherein abiraterone acetate is administered orally to the male human at a dose of about 1000 mg per day.
   7. The method of any one of the preceding embodiments, wherein the corticosteroid is prednisone, a prednisolone, or dexamethasone.
   8. The method of any one of the preceding embodiments, wherein the corticosteroid is administered to the male human at a dose of about 5 mg twice per day.
   9. The method of any one of the preceding embodiments, wherein prior to administration of the compound of formula I and abiraterone acetate plus the corticosteroid, said male human has not been administered an androgen receptor signaling inhibitor.
   10. The method of embodiment 9, wherein the androgen receptor signaling inhibitor is apalutamide.
   11. The method of any one of the preceding embodiments, wherein prior to administration of the compound of formula I, and abiraterone acetate plus the corticosteroid, said male human has been administered docetaxel.
   12. The method of any one of the preceding embodiments, wherein prior to administration of the compound of formula I, and abiraterone acetate plus the corticosteroid, said male human has been diagnosed with non-metastatic castration-resistant prostate cancer, metastatic castration-resistant prostate cancer, metastatic castration-sensitive prostate cancer, or locally advanced prostate cancer.
   13. The method of any one of the preceding embodiments, wherein administration of the compound of formula I, abiraterone acetate and the corticosteroid provides a composite response rate in the male human.
   14. The method of embodiment 13, wherein the composite response rate comprises a PSA90, an objective response rate, or both.
   15. The method of embodiment 13 or 14, wherein the composite response rate is at 12 weeks post administration of the compound of formula I and apalutamide.
   16. A method of treating metastatic castration-resistant prostate cancer (mCRPC) in a male human, said method comprising administering a therapeutically effective amount of a compound of formula I and a therapeutically effective amount of apalutamide to said male human

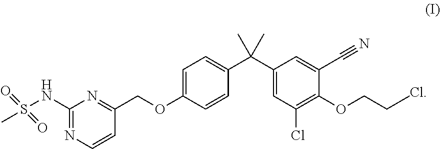

17. The method of embodiment 16, wherein the compound of formula I is administered orally to the male human at a dose of from about 600 mg per day to about 1000 mg per day.
18. The method of embodiment 16 or 17, wherein the compound of formula I is administered orally to the male human at a dose of:
(a) about 600 mg per day;
(b) about 800 mg per day; or
(c) about 1000 mg per day.
   19. The method of any one of embodiments 16 to 18, wherein the compound of formula I is administered orally to the male human at a dose of about 800 mg per day.
   20. The method of any one of embodiments 16 to 19, wherein apalutamide is administered orally to the male human at a dose of about 240 mg per day.
   21. The method of any one of embodiments 16 to 20, wherein prior to administration of the compound of formula I and abiraterone acetate plus the corticosteroid, said male human has not been administered an androgen receptor signaling inhibitor.
   22. The method of embodiment 21, wherein the androgen receptor signaling inhibitor is apalutamide.
   23. The method of any one of embodiments 16 to 22, wherein prior to administration of the compound of formula I and apalutamide, said male human has been administered docetaxel.
   24. The method of any one of embodiments 16 to 23, wherein prior to administration of the compound of formula I and apalutamide, said male human has been diagnosed with non-metastatic castration-resistant prostate cancer, metastatic castration-resistant prostate cancer, metastatic castration-sensitive prostate cancer, or locally advanced prostate cancer.
   25. The method of any one of embodiments 16 to 24, wherein administration of the compound of formula I and apalutamide provides a composite response rate in the male human.
   26. Th method of embodiment 25, wherein the composite response rate comprises a PSA90, an objective response rate, or both.
   27 The method of embodiment 25 or 26, wherein the composite response rate is at 12 weeks post administration of the compound of formula I and apalutamide.
   28. The method of any one of the preceding embodiments, administration of the compound of formula I is in further combination with androgen deprivation therapy
   29. The method of embodiment 28, wherein the androgen deprivation therapy consists of bilateral orchiectomy or gonadotropin-releasing hormone agonists or antagonists.
   30. A method of achieving a composite response rate in a male human with metastatic castration-resistant prostate cancer (mCRPC), said method comprising administering a therapeutically effective amount of the compound of formula I and abiraterone acetate plus a corticosteroid to said male human

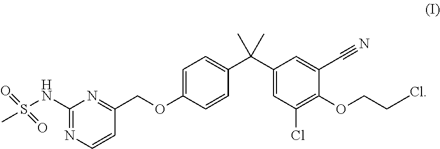

31. A method of achieving a composite response rate in a male human with metastatic castration-resistant prostate cancer (mCRPC), said method comprising administering a therapeutically effective amount of the compound of formula I and apalutamide to said male human

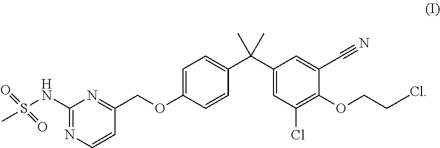

32. The method of embodiment 30 or 31, wherein the compound of formula I is administered orally to the male human at a dose of from about 600 mg per day to about 1000 mg per day.
33. The method of any one of embodiments 30 to 32, wherein the composite response rate comprises a PSA90, an objective response rate, or both.
34. The method of any one of embodiments 30 to 33, wherein the composite response rate is at 12 weeks post administration of the compound of formula I and apalutamide.

What is claimed:

1. A compound of formula I for use in a method of treating metastatic castration-resistant prostate cancer (mCRPC) in a male human, said method comprising administering a therapeutically effective amount of the compound of formula I and a therapeutically effective amount of abiraterone acetate plus a therapeutically effective amount of a corticosteroid to said male human

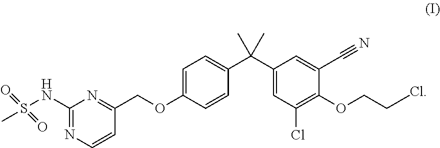

2. Abiraterone acetate for use in a method of treating metastatic castration-resistant prostate cancer (mCRPC) in a male human, said method comprising administering a therapeutically effective amount of abiraterone acetate plus a therapeutically effective amount of a corticosteroid and a therapeutically effective amount of a compound of formula I to said male human

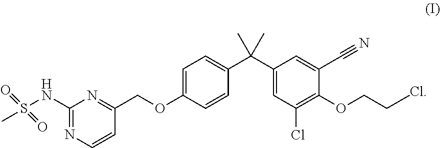

3. A corticosteroid for use in a method of treating metastatic castration-resistant prostate cancer (mCRPC) in a male human, said method comprising administering a therapeutically effective amount of the corticosteroid and a therapeutically effective amount of abiraterone acetate and a therapeutically effective amount of a compound of formula I to said male human

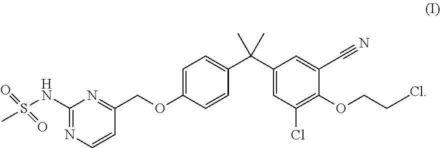

4. A compound of formula (I) and abiraterone acetate plus a corticosteroid for use in a method of treating metastatic castration-resistant prostate cancer (mCRPC) in a male human, said method comprising administering a therapeutically effective amount of the compound of formula I and a therapeutically effective amount of abiraterone acetate plus a therapeutically effective amount of the corticosteroid to said male human

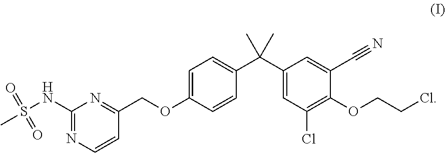 [SMILES: CC(C)(c1ccc(OCc2ccnc(NS(C)(=O)=O)n2)cc1)c1cc(Cl)c(OCCCl)c(C#N)c1]

5. A method of treating metastatic castration-resistant prostate cancer (mCRPC) in a male human, said method comprising administering a therapeutically effective amount of a compound of formula I and a therapeutically effective amount of abiraterone acetate plus a therapeutically effective amount of a corticosteroid to said male human

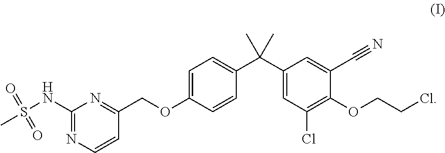 [SMILES: CC(C)(c1ccc(OCc2ccnc(NS(C)(=O)=O)n2)cc1)c1cc(Cl)c(OCCCl)c(C#N)c1]

6. The compound of formula (I) for use according to claim 1; abiraterone acetate for use according to claim 2; the corticosteroid for use according to claim 3; the compound of formula (I) and abiraterone acetate plus a corticosteroid for use according to claim 4; or the method of claim 5,
wherein the compound of formula I is administered orally to the male human at a dose of from about 600 mg per day to about 1000 mg per day.

7. The compound of formula (I) for use according to claim 1 or 6; abiraterone acetate for use according to claim 2 or 6; the corticosteroid for use according to claim 3 or 6; the compound of formula (I) and abiraterone acetate plus a corticosteroid for use according to claim 4 or 6; or the method of claim 5 or 6,
wherein the compound of formula I is administered orally to the male human at a dose of:
(a) about 600 mg per day;
(b) about 800 mg per day; or
(c) about 1000 mg per day.

8. The compound of formula (I) for use according to any one of claims 1 and 6-7; abiraterone acetate for use according to any one of claims 2 and 6-7; the corticosteroid for use according to any one of claims 3 and 6-7; the compound of formula (I) and abiraterone acetate plus a corticosteroid for use according to any one of claims 4 and 6-7; or the method of any one of claims 5-7,
wherein the compound of formula I is administered orally to the male human at a dose of about 800 mg per day.

9. The compound of formula (I) for use according to any one of claims 1 and 6-8; abiraterone acetate for use according to any one of claims 2 and 6-8; the corticosteroid for use according to any one of claims 3 and 6-8; the compound of formula (I) and abiraterone acetate plus a corticosteroid for use according to any one of claims 4 and 6-8; or the method of any one of claims 5-8,
wherein abiraterone acetate is administered orally to the male human at a dose of about 500 mg per day to about 1000 mg per day.

10. The compound of formula (I) for use according to any one of claims 1 and 6-9; abiraterone acetate for use according to any one of claims 2 and 6-9; the corticosteroid for use according to any one of claims 3 and 6-9; the compound of formula (I) and abiraterone acetate plus a corticosteroid for use according to any one of claims 4 and 6-9; or the method of any one of claims 5-9,
wherein abiraterone acetate is administered orally to the male human at a dose of about 1000 mg per day.

11. The compound of formula (I) for use according to any one of claims 1 and 6-10; abiraterone acetate for use according to any one of claims 2 and 6-10; the corticosteroid for use according to any one of claims 3 and 6-10; the compound of formula (I) and abiraterone acetate plus a corticosteroid for use according to any one of claims 4 and 6-10; or the method of any one of claims 5-10,
wherein the corticosteroid is prednisone, a prednisolone, or dexamethasone.

12. The compound of formula (I) for use according to any one of claims 1 and 6-11; abiraterone acetate for use according to any one of claims 2 and 6-11; the corticosteroid for use according to any one of claims 3 and 6-11; the compound of formula (I) and abiraterone acetate plus a corticosteroid for use according to any one of claims 4 and 6-11; or the method of any one of claims 5-11,
wherein the corticosteroid is prednisone.

13. The compound of formula (I) for use according to any one of claims 1 and 6-11; abiraterone acetate for use according to any one of claims 2 and 6-11; the corticosteroid for use according to any one of claims 3 and 6-11; the compound of formula (I) and abiraterone acetate plus a corticosteroid for use according to any one of claims 4 and 6-11; or the method of any one of claims 5-11,
wherein the corticosteroid is prednisolone.

14. The compound of formula (I) for use according to any one of claims 1 and 6-11; abiraterone acetate for use according to any one of claims 2 and 6-11; the corticosteroid for use according to any one of claims 3 and 6-11; the compound of formula (I) and abiraterone acetate plus a corticosteroid for use according to any one of claims 4 and 6-11; or the method of any one of claims 5-11,
wherein the corticosteroid is dexamethasone.

15. The compound of formula (I) for use according to any one of claims 1 and 6-14; abiraterone acetate for use according to any one of claims 2 and 6-14; the corticosteroid for use according to any one of claims 3 and 6-14; the compound of formula (I) and abiraterone acetate plus a corticosteroid for use according to any one of claims 4 and 6-14; or the method of any one of 5-14,
wherein the corticosteroid is administered to the male human at a dose of about 5 mg twice per day.

16. The compound of formula (I) for use according to claim 14; abiraterone acetate for use according to claim 14; the corticosteroid for use according to claim 14; the compound of formula (I) and abiraterone acetate plus a corticosteroid for use according to claim 14; or the method of claim 14,
wherein dexamethasone is administered to the male human at a dose of about 0.5 mg to about 1.0 mg once daily, twice daily or three times daily.

17. The compound of formula (I) for use according to claim 16; abiraterone acetate for use according to claim 16; the corticosteroid for use according to claim 16; the compound of formula (I) and abiraterone acetate plus a corticosteroid for use according to claim 16; or the method of claim 16,
wherein dexamethasone is administered to the male human at a dose of about 1.0 mg three times daily.

18. The compound of formula (I) for use according to any one of claims 1 and 6-17; abiraterone acetate for use according to any one of claims 2 and 6-17; the corticosteroid for use according to any one of claims 3 and 6-17; the compound of formula (I) and abiraterone acetate plus a corticosteroid for use according to any one of claims 4 and 6-17; or the method of any one of claims 5-17,
wherein prior to administration of the compound of formula I and abiraterone acetate plus the corticosteroid, said male human has not been administered an androgen receptor signaling inhibitor.

19. The compound of formula (I) for use according to claim 18; abiraterone acetate for use according to claim 18; the corticosteroid for use according to claim 18; the compound of formula (I) and abiraterone acetate plus a corticosteroid for use according to claim 18; or the method of claim 18,
wherein the androgen receptor signaling inhibitor is apalutamide.

20. The compound of formula (I) for use according to any one of claims 1 and 6-19; abiraterone acetate for use according to any one of claims 2 and 6-19; the corticosteroid for use according to any one of claims 3 and 6-19; the compound of formula (I) and abiraterone acetate plus a corticosteroid for use according to any one of claims 4 and 6-19; or the method of any one of claims 5-19,
wherein prior to administration of the compound of formula I, and abiraterone acetate plus the corticosteroid, said male human has been administered docetaxel.

21. The compound of formula (I) for use according to any one of claims 1 and 6-20; abiraterone acetate for use according to any one of claims 2 and 6-20; the corticosteroid for use according to any one of claims 3 and 6-20; the compound of formula (I) and abiraterone acetate plus a corticosteroid for use according to any one of claims 4 and 6-20; or the method of any one of claims 5-20,
wherein prior to administration of the compound of formula I, and abiraterone acetate plus the corticosteroid, said male human has been diagnosed with non-metastatic castration-resistant prostate cancer, metastatic castration-resistant prostate cancer, metastatic castration-sensitive prostate cancer, or locally advanced prostate cancer.

22. The compound of formula (I) for use according to any one of claims 1 and 6-21; abiraterone acetate for use according to any one of claims 2 and 6-21; the corticosteroid for use according to any one of claims 3 and 6-21; the compound of formula (I) and abiraterone acetate plus a corticosteroid for use according to any one of claims 4 and 6-21; or the method of any one of claims 5-21,
wherein administration of the compound of formula I, abiraterone acetate and the corticosteroid provides a composite response rate in the male human.

23. The compound of formula (I) for use according to claim 22; abiraterone acetate for use according to claim 22; the corticosteroid for use according to claim 22; the compound of formula (I) and abiraterone acetate plus a corticosteroid for use according to claim 22; or the method of claim 22,
wherein the composite response rate comprises a PSA90, an objective response rate, or both.

24. The compound of formula (I) for use according to claim 22 or 23; abiraterone acetate for use according to claim 22 or 23; the corticosteroid for use according to claim 22 or 23; the compound of formula (I) and abiraterone acetate plus a corticosteroid for use according to claim 22 or 23; or the method of claim 22 or 23,
wherein the composite response rate is at 12 weeks post administration of the compound of formula I and abiraterone acetate plus a corticosteroid.

25. A compound of formula I for use in a method of treating metastatic castration-resistant prostate cancer (mCRPC) in a male human, said method comprising administering a therapeutically effective amount of the compound of formula I and a therapeutically effective amount of apalutamide to said male human

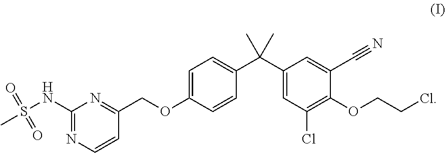 [SMILES: CC(C)(c1ccc(OCc2ccnc(NS(C)(=O)=O)n2)cc1)c1cc(Cl)c(OCCCl)c(C#N)c1]

26. Apalutamide for use in a method of treating metastatic castration-resistant prostate cancer (mCRPC) in a male human, said method comprising administering a therapeutically effective amount of apalutamide and a therapeutically effective amount of a compound of formula I to said male human

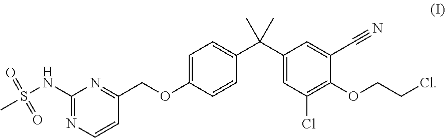 [SMILES: CC(C)(c1ccc(OCc2ccnc(NS(C)(=O)=O)n2)cc1)c1cc(Cl)c(OCCCl)c(C#N)c1]

27. A compound of formula I and apalutamide for use in a method of treating metastatic castration-resistant prostate cancer (mCRPC) in a male human, said method comprising administering a therapeutically effective amount of the compound of formula I and a therapeutically effective amount of apalutamide to said male human

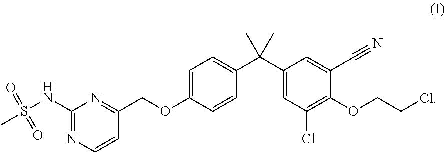 [SMILES: CC(C)(c1ccc(OCc2ccnc(NS(C)(=O)=O)n2)cc1)c1cc(Cl)c(OCCCl)c(C#N)c1]

28. A method of treating metastatic castration-resistant prostate cancer (mCRPC) in a male human, said method comprising administering a therapeutically effective amount of a compound of formula I and a therapeutically effective amount of apalutamide to said male human

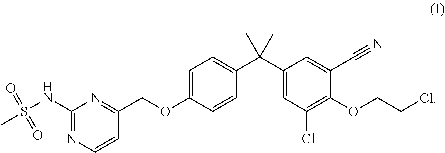 [SMILES: CC(C)(c1ccc(OCc2ccnc(NS(C)(=O)=O)n2)cc1)c1cc(Cl)c(OCCCl)c(C#N)c1]

29. The compound of formula I for use according to claim 25; apalutamide for use according to claim 26; the compound of formula I and apalutamide for use according to claim 27; or the method of claim 28,
wherein the compound of formula I is administered orally to the male human at a dose of from about 600 mg per day to about 1000 mg per day.

30. The compound of formula I for use according to claim 25 or 29; apalutamide for use according to claim 26 or 29; the compound of formula I and apalutamide for use according to claim 27 or 29; or the method of claim 28 or 29,
wherein the compound of formula I is administered orally to the male human at a dose of:
(a) about 600 mg per day;
(b) about 800 mg per day; or
(c) about 1000 mg per day.

31. The compound of formula I for use according to any one of claims 25, 29 and 30; apalutamide for use according to any one of claims 26, 29 and 30; the compound of formula I and apalutamide for use according to any one of claim 27, 29 or 30; or the method of any one of claims 28-30,
wherein the compound of formula I is administered orally to the male human at a dose of about 800 mg per day.

32. The compound of formula I for use according to any one of claims 25 and 29-31; apalutamide for use according to any one of claims 26 and 29-31; the compound of formula I and apalutamide for use according to any one of claims 27 and 29-31; or the method of any one of claims 28-31,
wherein apalutamide is administered orally to the male human at a dose of about 240 mg per day.

33. The compound of formula I for use according to any one of claims 25 and 29-32; apalutamide for use according to any one of claims 26 and 29-32; the compound of formula I and apalutamide for use according to any one of claims 27 and 29-32; or the method of any one of claims 28-32,
wherein prior to administration of the compound of formula I and apalutamide, said male human has not been administered an androgen receptor signaling inhibitor.

34. The compound of formula I for use according to claim 33; apalutamide for use according to claim 33; the compound of formula I and apalutamide for use according to claim 33; or the method of claim 33,
wherein the androgen receptor signaling inhibitor is apalutamide.

35. The compound of formula I for use according to any one of claims 25 and 29-34; apalutamide for use according to any one of claims 26 and 29-34; the compound of formula I and apalutamide for use according to any one of claims 27 and 29-34; or the method of any one of claims 28-34,
wherein prior to administration of the compound of formula I and apalutamide, said male human has been administered docetaxel.

36. The compound of formula I for use according to any one of claims 25 and 29-35; apalutamide for use according to any one of claims 26 and 29-35; the compound of formula I and apalutamide for use according to any one of claims 27 and 29-35; or the method of any one of claims 28-35,
wherein prior to administration of the compound of formula I and apalutamide, said male human has been diagnosed with non-metastatic castration-resistant prostate cancer, metastatic castration-resistant prostate cancer, metastatic castration-sensitive prostate cancer, or locally advanced prostate cancer.

37. The compound of formula I for use according to any one of claims 25 and 29-36; apalutamide for use according to any one of claims 26 and 29-36; the compound of formula I and apalutamide for use according to any one of claims 27 and 29-36; or the method of any one of claims 28-36,
wherein administration of the compound of formula I and apalutamide provides a composite response rate in the male human.

38. The compound of formula I for use according to claim 37; apalutamide for use according to claim 37; the compound of formula I and apalutamide for use according to claim 37; or the method of claim 37,
wherein the composite response rate comprises a PSA90, an objective response rate, or both.

39. The compound of formula I for use according to claim 37 or 38; apalutamide for use according to claim 37 or 38; the compound of formula I or apalutamide for use according to claim 37 or 38; or the method of claim 37 or 38,
wherein the composite response rate is at 12 weeks post administration of the compound of formula I and apalutamide.

40. The compound of formula I for use according to any one of claims 1, 6-25 and 29-39; abiraterone acetate for use according to any one of claims 2 and 6-24; the corticosteroid for use according to any one of claims 3 and 6-24; the compound of formula I, abiraterone acetate and the corticosteroid for use according to any one of claims 4 and 6-24; apalutamide for use according to any one of claims 26 and 29-39; the compound of formula I and apalutamide for use according to any one of claims 27 and 29-39; or the method of any one of claims 5-24 and 28-39,
wherein administration of the compound of formula I is in further combination with androgen deprivation therapy.

41. The compound of formula I for use according to claim 40; abiraterone acetate for use according to claim 40; the corticosteroid for use according to claim 40; the compound of formula I, abiraterone acetate and the corticosteroid for use according to claim 40; apalutamide for use according to claim 40; the compound of formula I and apalutamide for use according to claim 40; or the method of claim 40,
wherein the androgen deprivation therapy consists of bilateral orchiectomy or gonadotropin-releasing hormone agonists or antagonists.

42. A compound of formula I for use in a method of achieving a composite response rate in a male human with metastatic castration-resistant prostate cancer (mCRPC), said method comprising administering a therapeutically effective amount of the compound of formula I and abiraterone acetate plus a corticosteroid to said male human

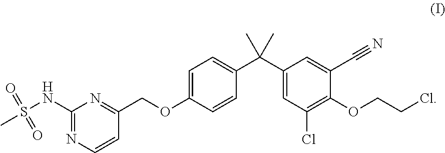 [SMILES: CC(C)(c1ccc(OCc2ccnc(NS(C)(=O)=O)n2)cc1)c1cc(Cl)c(OCCCl)c(C#N)c1]

43. Abiraterone acetate for use in a method of achieving a composite response rate in a male human with metastatic castration-resistant prostate cancer (mCRPC), said method comprising administering a therapeutically effective amount of abiraterone acetate plus a corticosteroid and a compound of formula I to said male human

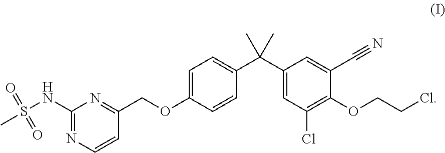 [SMILES: CC(C)(c1ccc(OCc2ccnc(NS(C)(=O)=O)n2)cc1)c1cc(Cl)c(OCCCl)c(C#N)c1]

44. A corticosteroid for use in a method of achieving a composite response rate in a male human with metastatic castration-resistant prostate cancer (mCRPC), said method comprising administering a therapeutically effective amount of the corticosteroid and abiraterone acetate and a compound of formula I to said male human

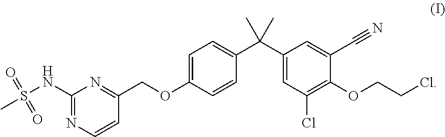 [SMILES: CC(C)(c1ccc(OCc2ccnc(NS(C)(=O)=O)n2)cc1)c1cc(Cl)c(OCCCl)c(C#N)c1]

45. A compound of formula I, abiraterone acetate and a corticosteroid for use in a method of achieving a composite response rate in a male human with metastatic castration-resistant prostate cancer (mCRPC), said method comprising administering a therapeutically effective amount of the compound of formula I and abiraterone acetate plus the corticosteroid to said male human

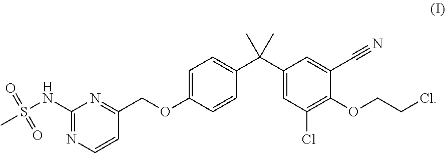 [SMILES: CC(C)(c1ccc(OCc2ccnc(NS(C)(=O)=O)n2)cc1)c1cc(Cl)c(OCCCl)c(C#N)c1]

46. A method of achieving a composite response rate in a male human with metastatic castration-resistant prostate cancer (mCRPC), said method comprising administering a therapeutically effective amount of the compound of formula I and abiraterone acetate plus a corticosteroid to said male human

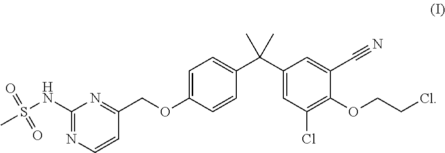 [SMILES: CC(C)(c1ccc(OCc2ccnc(NS(C)(=O)=O)n2)cc1)c1cc(Cl)c(OCCCl)c(C#N)c1]

47. A compound of formula I for use in a method of achieving a composite response rate in a male human with metastatic castration-resistant prostate cancer (mCRPC), said method comprising administering a therapeutically effective amount of the compound of formula I and apalutamide to said male human

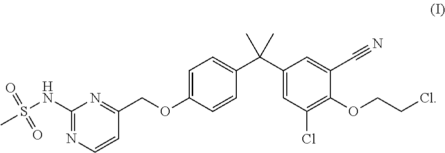 [SMILES: CC(C)(c1ccc(OCc2ccnc(NS(C)(=O)=O)n2)cc1)c1cc(Cl)c(OCCCl)c(C#N)c1]

48. Apalutamide for use in a method of achieving a composite response rate in a male human with metastatic castration-resistant prostate cancer (mCRPC), said method comprising administering a therapeutically effective amount of apalutamide and a compound of formula I to said male human

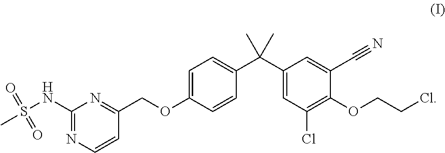 [SMILES: CC(C)(c1ccc(OCc2ccnc(NS(C)(=O)=O)n2)cc1)c1cc(Cl)c(OCCCl)c(C#N)c1]

49. A compound of formula I and apalutamide for use in a method of achieving a composite response rate in a male human with metastatic castration-resistant prostate cancer (mCRPC), said method comprising administering a therapeutically effective amount of the compound of formula I and apalutamide to said male human

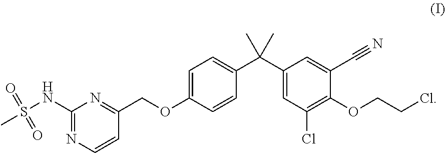 [SMILES: CC(C)(c1ccc(OCc2ccnc(NS(C)(=O)=O)n2)cc1)c1cc(Cl)c(OCCCl)c(C#N)c1]

50. A method of achieving a composite response rate in a male human with metastatic castration-resistant prostate cancer (mCRPC), said method comprising administering a therapeutically effective amount of the compound of formula I and apalutamide to said male human

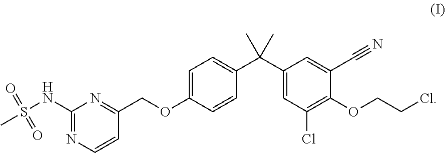 [SMILES: CC(C)(c1ccc(OCc2ccnc(NS(C)(=O)=O)n2)cc1)c1cc(Cl)c(OCCCl)c(C#N)c1]

51. The compound of formula I for use according to claim 42 or 47; abiraterone acetate for use according to claim 43; the corticosteroid for use according to claim 44; the compound of formula I, abiraterone acetate and the corticosteroid for use according to claim 45; apalutamide for use according to claim 48; the compound of formula I and apalutamide for use according to claim 49; or the method of claim 46 or 50,
wherein the compound of formula I is administered orally to the male human at a dose of from about 600 mg per day to about 1000 mg per day.

52. The compound of formula I for use according to any one of claims 42, 47 and 51; abiraterone acetate for use according to claim 43 or 51; the corticosteroid for use according to claim 44 or 51; the compound of formula I, abiraterone acetate and the corticosteroid for use according to claim 45 or 51; apalutamide for use according to claim 48 or 51; the compound of formula I and apalutamide for use according to claim 49 or 51; or the method of any one of claims 46, 50 and 51,
wherein the composite response rate comprises a PSA90, an objective response rate, or both.

53. The compound of formula I for use according to any one of claims 42, 47 and 51-52; abiraterone acetate for use according to any one of claims 43 and 51-52; the corticosteroid for use according to any one of claims 44 and 51-52; the compound of formula I, abiraterone acetate and the corticosteroid for use according to any one of claims 45 and 51-52; apalutamide for use according to any one of claims 48 and 51-52; the compound of formula I and apalutamide for use according to any one of claims 49, 51 and 52; or the method of any one of claims 46 and 50-52,
wherein the composite response rate is at 12 weeks post administration.
